SEQUENCE LISTING (33 sequences)

SEQ ID NO: 1 - protein, 23 aa, Mus Musculus
DETIEIIVENKVKELLANPANYP

SEQ ID NO: 2 - DNA, 27 bp, Artificial sequence
  sequence is synthesized
acaaacgcgtgaygaracnathgarat

SEQ ID NO: 3 - DNA, 27 bp, Artificial sequence
  sequence is synthesized
tggtgcatgcggrtarttngcnggrtt

SEQ ID NO: 4 - DNA, 34 bp, Mus Musculus
tatcgtggagaataaggtcaaggaacttcttgcc

SEQ ID NO: 5 - DNA, 34 bp, Mus Musculus
atagcacctcttattccagttccttgaagaacgg

SEQ ID NO: 6 - protein, 11 aa, Mus Musculus
IVENKVKELLA

SEQ ID NO: 7 - DNA, 30 bp, Mus Musculus
acaaacgcgtgctggagaataaggtcaagg

SEQ ID NO: 8 - DNA, 30 bp, Mus Musculus
actaacgcgtaggctaaggaacttcttgcc

SEQ ID NO: 9 - DNA, 536 bp, Mus Musculus
  unsure 389, 447: unknown base
ccgggccccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccanctttgatatgatgaatctaacaaaaactgcagtctcaatttggaaatctgactatgtncctttaaatgtgttcatattgcccatttaccctgcttcttgaaatgcttcttgaaaaataagacaattgcatgtgtaaaaaaaaaaaaa

SEQ ID NO: 10 - protein, 111 aa, Mus Musculus
MKTTTCSLLICISLLQLMVPVNTDETIEIIVENKVKELLANPANYPSTVTKTLSCTSVKTMNRWASCPAGMTATGCACGFACGSWEIQSGDTCNCLCLLVDWTTARCCQLS

SEQ ID NO: 11 - DNA, 50 bp, Mus Musculus
atctgttcatagtcttgacactagtgcaagagagagtcttcgttacagtg

SEQ ID NO: 12 - DNA, 1097 bp, Artificial sequence
  sequence is synthesized
gccctttcgtcttcaagaattcatgctgtggtgtcatgktcggtgatcgccagggtgccgacgccatcttgactgcaggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatctagaattatgaaaaagaatatcgcacaccatcaccatcaccatcaccatgcatcagatgacgatgacaaagacgaaaccatcgagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccagctttgatatgatgaatctaacaaaaactgcagtctcaatttggaaatctgactcatgtgcctttaaatgtgttcatattgcccatttaccctgcttcttgaaatgcttcttgaaaaataaagacaaatttgcatgtggaaaaaaaaaaaaaaaaagcatgcaccattccttgcggcggcggtgctcaacggcctcaacctactactgggctgcttcctaatgcaggagtcgcataagggagagcgtcgaccgatgcccttgagagccttcaacccagtcagctcccttccggtgggcgcggggcatgactatcgtccgccgcacttatgactgtcttctttttcatgcaactcgtaggacaggtgccggcagcgctcctgggtcattttcggcraggaccgctttcgctggagcgcgacgatgatcggcctgtcgc

SEQ ID NO: 13 - DNA, 523 bp, Mus Musculus
attcggatccaaccctgagctttctggagagtgaatctgctcttaggggaaaagctcttccctttccttctccaaaaagctagaactgagctccaggaggctgactttctacagcatgaagcctacactgtgtttccttttcatcctcgtctcccttttcccactgatagtcccagggaacgcgcaatgctcctttgagtctttggtggatcaaaggatcaaggaagctctcagtcgtcaagagcctaagacgatctcctgcactagtgtcacgtcttctggcagactggcctcctgtcctgctgggatggttgtcactggatgtgcttgtggctatggctgtggatcgtgggatatccggaatggaaatacttgccactgccagtgctcagtcatggactgggcctctgccgctgctgccgaatggttaagaatgaggaggttgagaaaccaatttcaaaatgatgagcataatgaaaccacggtctcgaccaggaaacctgactcattgtcttcatattac

SEQ ID NO: 14 - protein, 105 aa, Mus Musculus
MKPTLCFLFILVSLFPLIVPGNAQCSFESLVDQRIKEALSRQEPKTISCTSVTSSGRLASCPAGMVVTGCACGYGCGSWDIRNGNTCHCQCSVMDWASARCCRMA

SEQ ID NO: 15 - DNA, 372 bp, Mus Musculus
gggacaggagctaatacccagaactgagttgtgtcctgctaagtcctctgccacgtacccacgggatgaagaacctttcatttcccctccttttccttttcttccttgtccctgaactgctgggctccagcatgccactgtgtcccatcgatgaagccatcgacaagaagatcaaacaagacttcaactccctgtttccaaatgcaataaagaacattggcttaaattgctggacagtctcctccagagggaagttggcctcctgcccagaaggcacagcagtcttgagctgctcctgtggctctgcatgtgcgtcgtgggacattcgtgaagaaaaagtgtgtcactgccagtgtgcaaggatagactgga

SEQ ID NO: 16 - protein, 114 aa, Mus Musculus
MKNLSFPLLFLFFLVPELLGSSMPLCPIDEAIDKKIKQDFNSLFPNAIKNIGLNCWTVSSRGKLASCPEGTAVLSCSCGSACASWDIREEKVCHCQCARIDWTAARCCKLQVAS

SEQ ID NO: 17 - DNA, 703 bp, Homo sapien
tctgaatgttttggtgaataaatctgttcttcagcaaccctacctgcttctccaaactgcctaaagagatccagtactgatgacgctgttcttccatctttactccctggaaactaaccacgttgtcttctttccttcaccaccacccaggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaagacaaactttgtacctctcaaaaaaaaaaa

SEQ ID NO: 18 - protein, 111 aa, Homo sapien
MGPSSCLLLILIPLLQLINPGSTQCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 19 - DNA, 476 bp, Homo sapien
  unsure 389: unknown base
gtgtgccggatttggttagctgagcccaccgagaggcgcctgcaggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagtgcnacagcgcgggcggaggcggctccaggtccggaggggttgcgggggagctggaaataaacctggagatgatgatgatgatgatgatgg

SEQ ID NO: 20 - DNA, 523 bp, Mus Musculus; identical to SEQ ID NO: 13

SEQ ID NO: 21 - DNA, 372 bp, Mus Musculus; identical to SEQ ID NO: 15

SEQ ID NO: 22 - DNA, 577 bp, Homo sapien
tgaggtacaaagtttgtctttattacccaagaatcaggaatggaacaaatgaagtgggacgtttgagttagatttcttggttgggaccctggtttcattactgtcatggtcacaaaactgagttctcagcctcctccctgtcaggtcaggtggcagcagcgggcagtggtccagtccaccacactgcactggcagtggcaggtggtttccagctgaacatcccacgaaccacagccatagccacaagcacagccagtgacagccatcccagcagggcagtgaggacggtctgccttggcttttgacactagcacacgagagcttcttgcttataggagagggactgtactctagactgttgagaacatccttgatcttcttatccataacggagtctaaggaacactgagtactccccgggttgatcagctggagaagggggattaggatgagaaggaggcaagaggacggccccatcctgtacagagtcagtgtcctggggctgggggaaagatggaaagagcttagatctctgagccctgggtggtggtgaggaaagaagacacgtggctcgtgc

SEQ ID NO: 23 - DNA, 462 bp, Homo sapien
agcccaccgagaggcgcctgcaggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatggaaaaa

SEQ ID NO: 24 - protein, 108 aa, Homo sapien
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 25 - DNA, 27 bp, Homo sapien
ggatttggttagctgagcccaccgaga

SEQ ID NO: 26 - DNA, 26 bp, Homo sapien
gcactgcgcgcgacctcagggctgca

SEQ ID NO: 27 - DNA, 50 bp, Homo sapien
cttattgccctaaatattagggagccggcgacctcctggatcctctcatt

SEQ ID NO: 28 - DNA, 223 bp, Mus Musculus
cccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcct

SEQ ID NO: 29 - DNA, 251 bp, Mus Musculus
ccctgagctttctggagagtgaatctgctcttagggaaaagctcttccctttccttctccaaaaagctagaactgagctccaggaggctgactttctacagcatgaagcctacactgtgtttccttttcatcctcgtctcccttttcccactgatagtcccagggaacgcgcaatgctcctttgagtctttggtggatcaaaggatcaaggaagctctcagtcgtcaagagcctaagacgatctcctgcac

SEQ ID NO: 30 - DNA, 328 bp, Mus Musculus
cgagggggacaggagctaatacccagaactgagttgtgtcctgctaagtcctctgccacgtacccacgggatgaagaacctttcatttcccctccttttccttttcttccttgtccctgaactgctgggctccagcatgccactgtgtcccatcgatgaagccatcgacaagaagatcaaacaagacttcaactccctgtttccaaatgcaataaagaacattggcttaaattgctggacagtctcctccagagggaagttggcctcctgcccagaaggcacagcagtcttgagctgctcctgtggctctgcctgtggctcgtgggac

SEQ ID NO: 31 - DNA, 536 bp, Mus Musculus
  unsure 90, 148: unknown base
tttttttttttttacacatgcaattgtcttatttttcaagaagcatttcaagaagcagggtaaatgggcaatatgaacacatttaaaggnacatagtcagatttccaaattgagactgcagtttttgttagattcatcatatcaaagntgggttctccacctcttcattcttaggacagttggcagcagcgggcagtggtccagtcaacgagtaagcacaggcagttgcaagtatctccactctggatctcccaagatccacaggcaaagccacaagcacacccagtagcagtcatcccagcagggcaggaggcccatctgttcatagtcttgacactagtgcaagagagagtcttcgttacagtggagggatagttagctggattggcaagaagttccttgaccttattctccacgataatctctatggtctcatcagtattcactgggaccatcagctggagcagggagatgcagatgagaagggaacaagttgtagtcttcatcctattcaaagttggcatcctggggcccgg

SEQ ID NO: 32 - DNA, 1097 bp, Mus Musculus
gcgacaggccgatcatcgtcgcgctccagcgaaagcggtcctygccgaaaatgacccaggagcgctgccggcacctgtcctacgagttgcatgaaaaagaagacagtcataagtgcggcggacgatagtcatgccccgcgcccaccggaagggagctgactgggttgaaggctctcaagggcatcggtcgacgctctcccttatgcgactcctgcattaggaagcagcccagtagtaggttgaggccgttgagcaccgccgccgcaaggaatggtgcatgctttttttttttttttttccacatgcaaatttgtctttatttttcaagaagcatttcaagaagcagggtaaatgggcaatatgaacacatttaaaggcacatgagtcagatttccaaattgagactgcagtttttgttagattcatcatatcaaagctgggttctccacctcttcattcttaggacagttggcagcagcgggcagtggtccagtcaacgagtaagcacaggcagttgcaagtatctccactctggatctcccaagatccacaggcaaagccacaagcacacccagtagcagtcatcccagcagggcaggaggcccatctgttcatagtcttgacactagtgcaagagagagtcttcgttacagtggagggatagttagctggattggcaagaagttccttgaccttattctccacgataatctcgatggtttcgtctttgtcatcgtcatctgatgcatggtgatggtgatggtgatggtgtgcgatattctttttcataattctagataccctttttacgtgaacttgcgtactagttaactagttcgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcacctgcagtcaagatggcgtcggcaccctggcgatcaccgamcatgacaccacagcatgaattcttgaagacgaaagggc

SEQ ID NO: 33 - protein, 109 aa, Artificial sequence
  sequence is snythesized
MKKNIAHHHHHHHHASDDDDKDETIEIIVENKVKELLANPANYPSTVTKTLSCTSVKTMNRWASCPAGMTATGCACGFACGSWEIQSGDTCNCLCLLVDWTTARCCQLS

(19) United States
(12) Patent Application Publication
(10) Pub. No.: US 2004/0018980 A1
(43) Pub. Date: Jan. 29, 2004

(54) Novel FIZZ proteins

(75) Inventors: Austin L. Gurney, Belmont, CA (US); Caroline Hebert, Berkeley, CA (US); William J. Henzel, San Mateo, CA (US); Rhona Kabakoff, Pacifica, CA (US); David L. Shelton, Oakland, CA (US); Daniel B. Tumas, Orinda, CA (US)

Correspondence Address: GENENTECH, INC., 1 DNA WAY, SOUTH SAN FRANCISCO, CA, 94080, US

(73) Assignee: Genentech, Inc., South San Francisco, CA

(21) Appl. No.: 10/461,060

(22) Filed: Jun. 13, 2003

Related U.S. Application Data
(63) Continuation of application No. 09/380,913, filed on Sep. 9, 1999, now abandoned.
Application No. 09/380,913 is a 371 of international application No. PCT/US99/08615, filed on Apr. 20, 1999.
(60) Provisional application No. 60/082,999, filed on Apr. 24, 1998.
(60) Provisional application No. 60/085,149, filed on May 12, 1998.
(60) Provisional application No. 60/100,263, filed on Sep. 14, 1998.

(51) Int. Cl. A61K 38/17

(52) U.S. Cl. 514/12

(57) ABSTRACT

The present invention is directed to novel polypeptides, designated FIZZ, which are secreted low molecular weight molecules showing no significant sequence homology to any known protein, and nucleic acid sequences encoding such proteins. Also provided herein are vectors and host cells comprising those nucleic acid sequences, chimeric polypeptide molecules comprising the polypeptide of the present invention fused to heterologous polypeptide sequences, and antibodies which bind to the polypeptides of the present invention. Methods of using FIZZ polypeptides to treat various neurotrophin-related conditions are further provided.

### FIELD OF THE INVENTION

The present invention relates generally to the identification and recombinant production of certain novel polypeptides, designated herein as “FIZZ” (for “Found in Inflammation Zone”).

### BACKGROUND OF THE INVENTION

Secreted Proteins

Extracellular proteins play an important role in the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature [see, for example, Klein et al., Proc. Natl. Acad. Sci., 93:7108-7113 (1996); U.S. Pat. No. 5,536,637)].

Neurotrophic Factors

Neurotrophic factors, including neurotrophins, are known to control a number of aspects of the function of the peripheral and central nervous system, which, in turn, is capable of modulating the function of essentially all other organs. Accordingly, neurotrophins are of enormous biological significance.

Probably the best known neurotrophic factor, nerve growth factor (NGF) is a protein which has prominent effects on developing sensory and sympathetic neurons of the peripheral nervous system. NGF acts via specific cell surface receptors on responsive neurons to support neuronal survival, promote neurite outgrowth, and enhance neurochemical function. NGF actions are accompanied by alterations in neuronal membranes (Connolly et al., 1981, J. Cell. Biol. 90:176; Skaper and Varon, 1980, Brain Res. 197:379), in the state of phosphorylation of neuronal proteins (Yu, et al., 1980, J. Biol. Chem. 255:10481; Haleqoua and Patrick, 1980, Cell 22:571), and in the abundance of certain mRNAs and proteins likely to play a role in neuronal differentiation and function (Tiercy and Shooter, 1986, J. Cell. Biol. 103:2367).

Forebrain cholinergic neurons also respond to NGF and may require NGF for trophic support. (Hefti, 1986, J. Neurosci., 6:2155). Indeed, the distribution and ontogenesis of NGF and its receptor in the central nervous system (CNS) suggest that NGF acts as a target-derived neurotrophic factor for basal forebrain cholinergic neurons (Korsching, November/December 1986, Trends in Neuro. Sci., pp 570-573).

While a number of animal homologues to NGF have become known, it was not until 1989 that an apparently distinct neurotrophic growth factor was identified that nonetheless bears some homology to NGF (Leibrock et al., 1989, Nature 341:149). This factor, called brain-derived neurotrophic factor (BDNF), was purified from pig brain, and a partial amino acid sequence determined both from the N-terminal end and from fragments purified after cleavages. The overall amino acid sequence identity between NGF and BNDF (NT-2) is about 50%. In view of this finding, Leibrock et al. speculated that there was no reason to think that BDNF and NGF should be the only members of a family of neurotrophic factors having in common structural and functional characteristics.

Indeed, subsequently another novel neurotrophic factor closely related to βNGF and BDNF was discovered, or neurotrophin-3 (NT-3). (Hohn, et al., 1990, Nature 344:339; Maisonpierre, et al., 1990, Science 247:1446; Rosenthal, et al., 1990, Neuron 4:767). Both BDNF and NT-3 share approximately 50% of their amino acids with βNGF. High levels of mRNA coding for BDNF and NT-3 occur in the adult rodent brain. βNGF, BDNF, and NT-3 support survival of selected populations of chick and mammalian sensory neurons, suggesting independent roles in neuronal survival.

Neurotrophins-4 and -5 (NT-4, NT-5), have been added to the family (PCT publication WO92/05254, published Apr. 2, 1992); Hallbook, F. et al., Neuron 6, 845-858 [1991]; Berkemeier, L. R. et al., Neuron 7, 857-866 [1991]).

In addition to their well characterized effects in the peripheral nervous system, various members of the neurotrophin family have been shown to play important roles in modulating the adult central nervous system as well. For instance, NGF is required for the normal neurochemical differentiation of basal forebrain cholinergic neurons, and also normal memory capability (Chen et al., J. Neurosci. 17(19):7288-96 [1997]). It is also known that BDNF can change the phenomenon known as long term potentiation, which is thought to also be related to cognitive function (Kang et al., Neuron 19:653-654 [1997]). Neurotrophins can also modulate other aspects of other CNS neuron function, such as BDNF modulation of serotonergic neurons (Siuciak et al., Brain Res. 710(1-2):11-20 [1996]). Therefore, neurotrophins are potentially involved in many aspects of CNS normal function and pathology.

Neuronal survival and growth is also affected by growth factors for non-neuronal cells, including fibroblast growth factor (FGF), epidermal growth factor, and insulin-like growth factors. (Morrison, et al., 1987, Science 238:72; Walicke, 1988, J. Neurosci. 8:2618; Bhat, 1983, Dev. Brain Res. 11:315). Basic FGF (bFGF) supports initial survival and subsequent fiber outgrowth of dissociated rodent fetal neurons in culture. While neurons from many brain regions are affected, the proportion of neurons surviving varies among brain regions, suggesting that subpopulations of neurons are responsive to bFGF. (Morrison, et al., 1986, Proc. Natl. Acad. Sci. 83:7537; Walicke, et al., 1986, Proc. Natl. Acad. Sci. USA 83:3012). Since bFGF lacks a signal sequence typical for released proteins, and since bFGF levels present in the brain are much larger than those of βNGF and BDNF, it has been questioned whether bFGF plays a physiological role as neurotrophic factor and has been proposed that bFGF acts as “injury factor” released in events involving cellular destruction. (Thoenen, et al., 1987, Rev. Physiol. Biochem. Pharmacol. 109:145).

Another neurotrophic factor having potential therapeutic use for peripheral nervous system disorders, ciliary neurotrophic factor (CNTF), has been cloned and expressed. (Lin, et al., 1989, Science, 246:1023). CNTF, which was purified from adult rabbit sciatic nerves, acts on the peripheral nervous system and appears to be completely unrelated to NGF.

Pantropic neurotrophic factors which have multiple neurotrophic specificities are provided, for example, in PCT Publication WO 95/33829, published Dec. 14, 1995.

Similarly to other polypeptide growth factors, the neurotrophic factors affect their target cells through interactions with cell surface receptors. NGF is currently known to have two receptors, a low molecular weight (65-80 kDa) receptor, termed p75 (or LNGFR), and a large molecular weight (130-150 kDa) receptor, termed p140^{trkA}.

p75 is present in some NGF responsive cells. Its isolation from rat and human sources (Radeke, M. J. et al., Nature 325, 593-597 [1987]; Johnson, D. et al., Cell 47, 545-554 [1986]) showed that this molecule is a glycoprotein which contains less than 50 kDa of protein while the rest of its molecular weight is due to the presence of N- and O-linked carbohydrate residues. p75 contains a single transmembrane segment flanked by extracellular and intracellular domains. Its extracellular domain contains four negatively charged cysteine rich repeats with the following pattern: Cys-X₁₀₋₁₄-Cys-X₂-Cys-X₂-Cys-X₉₋₁₁-Cys-X₈-Cys. Other conserved residues in the repeats include glycine, threonine, proline, and tyrosine (Smith, C. A. et al., Science 248, 1019-1023 [1990]). p75 in most cells binds ¹²⁵I-NGF with a K_{d} of 10⁻⁹ M, and is, therefore, often referred to as the “low affinity” NGF receptor. p75 is structurally related to the tumor necrosis factor receptors (TNF-R1 and TNF-R2), the Fas antigen, the B-cell antigen CD40, the MRC OX-40 antigen, which is a marker of activated T cells of the CD4 phenotype; a cDNA (4-1BB) which encodes a protein of unknown function and is obtained from T-cell clones; and SFV-T2, an open reading frame in Shope fibroma virus. It has been suggested that, in addition to NGF, other neurotrophic factors, and in particular BDNF and NT-3, bind p75. According to a recent report (Rabizadeh et al., Science 261, 345-348 [1993]), expression of p75 NGFR induces neural cell death (apoptosis) constitutively.

Various mutagenesis studies have shown that the amino acid sequences critical for NGF binding are likely to be within the third and fourth cysteine-rich repeats (amino acids 80-160) of the human p75 NGFR extracellular domain. In a study in which the primary structure of human p75 NGFR was perturbed with the introduction of linker insertions and short deletions (Yan, H. and Chao, M. V., J. Biol. Chem. 266, 12099-12104 [1991]), the most dramatic effects were observed at amino acid positions 105 and 130, which are located within the third and fourth cysteine-rich repeats, respectively. The observation that amino acid 130 and the surrounding residues are important for NGF binding is in good agreement with the conservation of these residues in various animal species, e.g. rat, chick, mouse and human. Indeed, a p75 variant from which amino acids 118-142 were deleted did not bind NGF. In an independent study Erlcher, A. A. et al. (Proc. Natl. Acad. Sci. USA 88, 159-163 [1991]) found human p75 variants in which either the first cysteine-rich repeat or the first and part of the second cysteine-rich repeat sequences were removed to bind NGF. However, a deletion mutant lacking all four cysteine-rich sequences of the p75 NGFR was unable to bind NGF.

p140^{trkaA} (hereinafter referred to as TrkA) belongs to the superfamily of receptor tyrosine kinases, and has been identified on NGF responsive cells. This receptor contains a domain specifically binding NGF resulting in a ligand-dependent activation of the tyrosine kinase. Recent site-directed mutagenesis studies have shown that NGF variants can be made that virtually eliminate p75 binding without loss of function in NGF responsive neurons or PC12 cells (Ibanez, C. F. et al., Cell 69, 329-341 [1992]). These findings indicate that TrkA alone is sufficient to induce at least some neurotrophic responses in target cells. The role of p75 remains unclear.

Of the other neurotrophic factors, BDNF was shown to bind selectively to another tyrosine kinase receptor, TrkB (Squinto, S. P., Cell 65, 885-893 [1991]; Soppet, D. et al., Cell 65, 895-903 [1991]; Klein, R. et al., Cell 66, 395-403 [1991]), whereas NT-3 binds to another homolog, TrkC (Lambelle, F. et al., Cell 66, 967-979 [1991]). NT-4 and NT-5 have been shown to strongly stimulate TrkB, but they have not yet been found to have a unique Trk receptor of their own. NT-3, NT-4 and NT-5 all appear to bind TrkA with lower affinity than NGF, although their effect on this receptor is controversial.

Biological Role of Neurotrophic Factors

NGF supports neuronal survival, promotes neurite outgrowth and enhances neurochemical function. NGF actions are known to be accompanied by alterations in neuronal membranes, in the state of phosphorylation of neuronal proteins, and in the abundance of certain mRNAs and proteins likely to play a role in neuronal differentiation and function. While the neurons of the peripheral nervous system (PNS) respond to all known neurotrophic factors, not all neurons respond to each one. NGF-responsive PNS neurons include sympathetic neurons and certain kinds of sensory neurons. Of these, sympathetic neurons do not respond or respond only poorly to other neurotrophins, while the response of PC12 cells is limited to NGF. Cholinergic neurons of the basal forebrain in the central nervous system (CNS) also respond at least to NGF and BDNF. BDNF has also been shown to affect dopaminergic neurons and retinal ganglion cells. All NGF responsive cells express the TrkA receptor, which is the primary mediator of NGF's biological responses.

Increases in NGF during inflammation increase the sensitivity of primary nociceptors and this is largely responsible for inflammatory pain. It has also been shown that normal levels of NGF contribute to the maintenance of normal pain sensitivity. But these sensory nerve fibers contribute to much more than pain sensitivity. They are also crucial for normal airway responsiveness, and their removal leads to a lack of normal or pathological modulation of airway constriction. Likewise, upregulation of sensitivity of sensory nerve fibers leads to hyperreflexia in urinary bladder. Neurotrophins are also known to affect sympathetic neurons, which are crucially involved in pain responses, as well as airway responsiveness, vascular tone, bowel motility, and cardiac rhythm. It has been recently demonstrated that neurotrophins are crucial for the maintenance of normal function in adult motorneurons as well, which control all voluntary movement. NGF is currently being developed for the treatment of peripheral sensory neuropathies, common in diabetes (diabetic neuropathy) and AIDS.

So far there have been no reports of the identification of endogenous inhibitors of neurotrophin action.

### SUMMARY OF THE INVENTION

Applicants have identified a new family of secreted proteins, designated in the present application as “Found in Inflammation Zone” or “FIZZ” polypeptides, with no significant sequence homology to known proteins. More specifically, applicants identified, by gel electrophoresis, a secreted low molecular weight (8-9 kDa) protein that was expressed in the airways of asthmatic mice, but not control mice, derived from a model of ovalbumin-induced asthma. With reference to the first three amino acids at the N-terminus of the mature murine protein (D, E and T), this molecule was originally designated as “m-DET1” (DNA53517), and is now referred to as “m-FIZZ1”. Two further mouse and two human homologs of m-FIZZ1 have been identified by homology searches in public databases, resulting in a family of five novel FIZZ proteins.

It has been found that the FIZZ proteins are capable of inhibiting the actions of neurotrophins on responsive neurons, and thus are the first known endogenous inhibitors of neurotrophin action.

In one aspect, the invention concerns isolated FIZZ polypeptides. In a particular embodiment, the invention concerns an isolated polypeptide comprising a FIZZ polypeptide sequence encoded by DNA having at least 80% sequence identity to a DNA molecule encoding amino acid residues 24-117 of FIG. 5 (SEQ ID NO: 10), or amino acid residues 21-105 of FIG. 10 (SEQ ID NO: 14), or amino acid residues 21 to 114 of FIG. 12 (SEQ ID NO: 16), or amino acid residues 21-111 of FIG. 14 (SEQ ID NO: 18), or amino acid residues 19 to 108 of FIG. 26 (SEQ ID NO: 24). In another embodiment, the isolated polypeptide comprises a FIZZ sequence encoded by DNA hybridizing under stringent conditions to the complement of a DNA molecule of FIG. 5 (SEQ ID NO: 9), FIG. 9 (SEQ ID NO: 13), FIG. 11 (SEQ ID NO: 15), FIG. 13 (SEQ ID NO: 17), FIG. 15 (SEQ ID NO: 19), or FIG. 25 (SEQ ID NO: 23). In a specific embodiment, the isolated FIZZ polypeptide is selected from the group consisting of m-FIZZ1 comprising amino acid residues 24-117 of FIG. 5 (SEQ ID NO: 10), m-FIZZ2 comprising amino acid residues 21-105 of FIG. 10 (SEQ ID NO: 14), m-FIZZ3 comprising amino acid residues 21-114 of FIG. 12 (SEQ ID NO: 16), h-FIZZ1 comprising amino acid residues 21-111 of FIG. 14 (SEQ ID NO: 18), and h-FIZZ3 comprising amino acid residues 19 to 108 of FIG. 26 (SEQ ID NO: 24). The FIZZ polypeptides may comprise an N-terminal signal peptide, which may, for example, be a signal peptide of a native FIZZ protein, or a heterologous signal, and may be fused or otherwise linked to other heterologous sequences, e.g., to a toxin moiety.

In another aspect, the invention concerns a chimeric molecule comprising a FIZZ polypeptide fused to a heterologous amino acid sequence. The heterologous amino acid sequence may, for example, be an epitope tag sequence, a Fc region of an immunoglobulin, or a toxin.

In yet another aspect, the invention concerns an antibody which specifically binds to a FIZZ polypeptide. The antibody can be an agonist, neutralizing or antagonist poly- or monoclonal antibody or antibody fragment.

In a further aspect, the invention concerns an isolated nucleic acid comprising DNA having at least a 80% sequence identity to (a) a DNA molecule encoding an m-FIZZ1 polypeptide having amino acid residues 24 to 111 of native m-FIZZ1 (FIG. 5, SEQ ID NO: 10), or (b) the complement of the DNA molecule of (a). In a particular embodiment, the isolated nucleic acid molecule comprises the DNA molecule of FIG. 5 (SEQ ID NO: 9). In another particular embodiment, the isolated nucleic acid molecule comprises the cDNA insert of the vector DNA53517-1366, deposited on Apr. 23, 1998 as ATCC No. 209802.

In a still further aspect, the invention concerns an expression vector comprising and capable of expressing a DNA having at least 80% sequence identity to a DNA molecule encoding amino acid residues 24-117 of FIG. 5 (SEQ ID NO: 10), or amino acid residues 21-105 of FIG. 10 (SEQ ID NO: 14), or amino acid residues 21 to 114 of FIG. 12 (SEQ ID NO: 16), or amino acid residues 21-111 of FIG. 14 (SEQ ID NO: 18), or amino acid residues 19 to 108 of FIG. 26 (SEQ ID NO: 24).

In yet another aspect, the invention concerns an expression vector comprising and capable of expressing a DNA hybridizing under stringent conditions to the complement of a DNA molecule of FIG. 5 (SEQ ID NO: 9), FIG. 9 (SEQ ID NO: 13), FIG. 11 (SEQ ID NO: 15), FIG. 13 (SEQ ID NO: 17), FIG. 15 (SEQ ID NO: 19), or FIG. 25 (SEQ ID NO: 23).

The invention also concerns host cells transformed with the expression vectors above. The host cells may be prokaryotic, e.g. E. coli, or eukaryotic, e.g. mammalian (such as, CHO, COS) or yeast (such as, Saccharomyces cerevisiae).

In another aspect, the invention concerns a process for producing a FIZZ polypeptide comprising culturing the host cells transformed with the expression vectors herein, under conditions suitable for expression of FIZZ and recovering the FIZZ polypeptide from the cell culture.

In yet another aspect, the present invention concerns a method of enhancing the immune response in a patient comprising administering to the patient an effective amount of a FIZZ protein or an agonist of a FIZZ protein. The FIZZ protein preferably is FIZZ1.

In a different aspect, the invention concerns a method of suppressing the immune response in a patient by administering to the patient an effective amount of an antagonist of a FIZZ protein, e.g. a small molecule antagonist or an anti-FIZZ antibody.

In a further aspect, the invention concerns a method of treating a pathologic condition associated with neurotrophin action on responsive neurons, comprising administering to a patient an effective amount of a FIZZ protein or an agonist of a FIZZ protein. In a still further aspect, the invention relates to a method of treating a pathologic condition associated with the neutrophin-inhibitory activity of a FIZZ polypeptide, comprising administering to a patient an antagonist of a FIZZ protein. In both methods, the agonist or antagonist may, for example, be anti-FIZZ antibody.

The invention further concerns a composition comprising a FIZZ polypeptide, or an agonist or antagonist of a FIZZ polypeptide, in combination with a carrier, optionally, a pharmaceutically-acceptable carrier.

The invention additionally concerns a method of screening for an antagonist or agonist of a FIZZ polypeptide, comprising contacting neurotrophin-responsive neurons, in the presence of a neurotrophin and a FIZZ polypeptide, with a candidate molecule, and monitoring neurotrophin action on the neurons, in comparison with neurotrophin action in the absence of the candidate molecule. The screening assays may be performed in a variety of formats, such as, for example, in the KIRA-ELISA format.

### BRIEF DESCRIPTION OF THE DRAWINGS

FIG. 1 Mouse Asthma Protocol showing immunization/aerosolization regimens for asthmatic and control mice.

FIG. 2 Silver stained 16% Tricine gel: Lane 1: control mouse BAL; Lane 2: asthmatic mouse BAL; lane 3: 8.3 kDa molecular weight marker (IL-8).

FIG. 3 Protein sequence analysis of a FIZZ sample blotted onto a PVDF membrane.

FIG. 4 Partial m-FIZZ1 cDNA sequence and corresponding amino acid sequence.

FIG. 5 Full length m-FIZZ1 cDNA sequence (SEQ ID NO: 9) and corresponding amino acid sequence (SEQ ID NO: 10). The amino acid sequence includes a putative signal peptide between residues 1-23, and a putative calcium-binding EGF-like domain protein pattern between residues 84-93.

FIG. 6 The FIZZ expression construct in pST31: DNA sequence (SEQ ID NO: 12) and corresponding amino acid sequence of the (His)8-tagged m-FIZZ1 fusion protein.

FIG. 7 Mouse tissue Northern blot probed with a radiolabelled m-FIZZ1 probe. Lanes from left to right: heart, brain, spleen, lung, liver, skeletal muscle, kidney, testis.

FIG. 8 In situ hybridization of mouse lung tissue sections probed with a radiolabelled m-FIZZ1 probe: (A) asthmatic mouse lung; (B) control mouse lung.

FIG. 9 Single-stranded nucleotide sequence encoding m-FIZZ2 (SEQ ID NO: 13).

FIG. 10 Amino acid sequence of m-FIZZ2 (SEQ ID NO: 14). The sequence includes a putative signal peptide between residues 1-20, and a putative prenyl group binding site (CAAX box) between residues 102-105.

FIG. 11 Single-stranded nucleotide sequence encoding m-FIZZ3 (SEQ ID NO: 15).

FIG. 12 Amino acid sequence of m-FIZZ3 (SEQ ID NO: 16). The sequence includes a putative signal peptide between residues 1-20, a putative leucine zipper pattern between residues 4-25, an N-glycosylation site starting at residue 3, and a sequence motif between residues 39-48, usually characteristic of DNA polymerase family B proteins.

FIG. 13 Single-stranded nucleotide sequence encoding h-FIZZ1 (SEQ ID NO: 17).

FIG. 14 Amino acid sequence of h-FIZZ1 (SEQ ID NO: 18). The sequence includes a putative signal peptide between residues 1-20, and a putative prenyl group binding site (CAAX box) between residues 108-111.

FIG. 15 Single-stranded nucleotide sequence of a virtual DNA encoding a human m-FIZZ homologue (SEQ ID NO: 19). EST AA311223, renamed as DNA53028.

FIG. 16 The nucleotide sequence of EST AA245405 (SEQ ID NO: 20).

FIG. 17 The nucleotide sequence of EST W42069 (SEQ ID NO: 21).

FIG. 18 The nucleotide sequence of EST AA524300 (SEQ ID NO: 22).

FIG. 19 Bar graphs illustrating that the addition of m-FIZZ1 to embryonic DRG cultures inhibits neuronal survival induced by a combination of neurotrophins (NGF, BDNF and NT3) in a dose dependent fashion.

FIG. 20 m-FIZZ1 inhibition of neuronal survival induced in DRG cultures by NGF alone or BDNF alone.

FIG. 21 m-FIZZ1 inhibition of the rise of NGF-induced CGRP content in adult DRG neuron cultures.

FIG. 22 Inhibition of NGF bioactivity by m-FIZZ3.

FIG. 23 Binding of NGF to human trkA-IgG in the absence or presence of m-FIZZ1.

FIG. 24 Alignment of the m-FIZZ1, m-FIZZ2, m-FIZZ3 and h-FIZZ1 proteins.

FIG. 25 Single-stranded nucleotide sequence encoding h-FIZZ3 (SEQ ID NO: 23).

FIG. 26 Amino acid sequence of hFIZZ3 (SEQ ID NO: 24). The sequence includes a putative signal sequence between residues 1 and 18, and a cell attachment sequence (RGD) starting at position 57.

FIG. 27 Alignment of the m-FIZZ1 and h-FIZZ3 proteins.

### DETAILED DESCRIPTION OF THE PREFERRED EMBODIMENTS

I. Definitions

The terms “FIZZ polypeptide”, “FIZZ protein” and “FIZZ” when used herein encompass native sequence FIZZ and FIZZ variants (which are further defined herein). The FIZZ polypeptide may be isolated from a variety of sources, such as from mouse or human tissue types or from another source, or prepared by recombinant or synthetic methods.

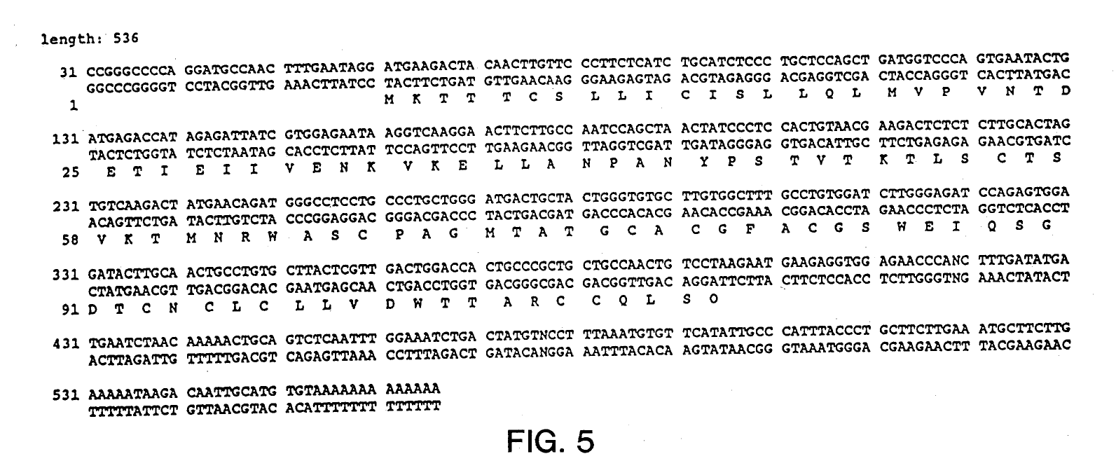

A “native sequence FIZZ” comprises a polypeptide having the same amino acid sequence as a FIZZ polypeptide derived from nature. Such native sequence FIZZ can be isolated from nature or can be produced by recombinant or synthetic means. The term “native sequence FIZZ” specifically encompasses naturally-occurring or truncated forms of the FIZZ proteins, naturally-occurring variant forms (e.g., alternatively spliced forms) and naturally-occurring allelic variants. In one embodiment of the invention, the native sequence FIZZ polypeptide is a mature or full-length native sequence murine FIZZ (m-FIZZ) comprising amino acid residues 1-117 of m-FIZZ1 (FIG. 5 SEQ ID NO: 10), or amino acid residues 1-105 of m-FIZZ2 (FIG. 10, SEQ ID NO: 14), or amino acid residues 1-114 of m-FIZZ3 (FIG. 12, SEQ ID NO: 16), or a fragment thereof, lacking the N-terminal signal peptide. In another embodiment, the native sequence FIZZ polypeptide is a mature or full-length native sequence human FIZZ (h-FIZZ) comprising amino acid residues 1-111 of h-FIZZ1 (FIG. 14, SEQ ID NO: 18), or amino acid residues 19-108 of h-FIZZ3 (FIG. 26 (SEQ ID NO: 24), or a fragment thereof, lacking the N-terminal signal peptide. It is noted that similar designations in murine and human FIZZ proteins, such as, m-FIZZ1/h-FIZZ1, or m-FIZZ3/h-FIZZ3 merely indicate that there is a high degree of sequence identity and structural similarity between the murine and human proteins designated using the same suffix. The use of the same suffix in a murine and human protein does not necessarily mean, however, that the human protein is the human homologue of the murine protein. It is possible, and contemplated, that further murine and human FIZZ proteins exist and can be identified, and the human proteins disclosed herein may be the homologues of other murine FIZZ proteins not yet identified.

“FIZZ variant” means an active FIZZ as defined below encoded by a nucleic acid comprising DNA having at least about 80% nucleic acid sequence identity to (a) a DNA molecule encoding a m-FIZZ1, m-FIZZ2, m-FIZZ3, h-FIZZ1 or h-FIZZ3 polypeptide, with or without the respective native signal sequences, or (b) the complement of a DNA molecule of (a). In a particular embodiment, the “FIZZ variant” has at least about 80% amino acid sequence identity with a full length native sequence m-FIZZ1, m-FIZZ2, m-FIZZ3, h-FIZZ1 or h-FIZZ3 polypeptide. Such FIZZ variants include, for example, FIZZ variant wherein one or more amino acid residues are added, or deleted, at the N- or C-terminus of a native sequence FIZZ polypeptide. Preferably, the nucleic acid or amino acid sequence identity is at least about 85%, more preferably at least about 90%, and even more preferably at least about 95%.

“Percent (%) amino acid sequence identity” with respect to the FIZZ sequences identified herein is defined as the percentage of amino acid residues in a candidate sequence that are identical with the amino acid residues in the FIZZ sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 [1996]; http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

“Percent (%) nucleic acid sequence identity” with respect to the FIZZ sequences identified herein is defined as the percentage of nucleotides in a candidate sequence that are identical with the nucleotides in the FIZZ sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 [1996]; http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

“Isolated,” when used to describe the various FIZZ polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the FIZZ natural environment will not be present. Ordinarily, however, isolated polypeptide will be prepared by at least one purification step.

An “isolated” FIZZ nucleic acid molecule is a nucleic acid molecule that is identified and separated from at least one contaminant nucleic acid molecule with which it is ordinarily associated in the natural source of the FIZZ nucleic acid. An isolated FIZZ nucleic acid molecule is other than in the form or setting in which it is found in nature. Isolated FIZZ nucleic acid molecules therefore are distinguished from the FIZZ nucleic acid molecule as it exists in natural cells. However, an isolated FIZZ nucleic acid molecule includes FIZZ nucleic acid molecules contained in cells that ordinarily express FIZZ where, for example, the nucleic acid molecule is in a chromosomal location different from that of natural cells.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment near but below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “expression vector” is used to define a vector, in which a nucleic acid encoding a FIZZ protein herein is operably linked to control sequences capable of affecting its expression is a suitable host cells. Vectors ordinarily carry a replication site (although this is not necessary where chromosomal integration will occur). Expression vectors also include marker sequences which are capable of providing phenotypic selection in transformed cells. For example, E. coli is typically transformed using pBR322, a plasmid derived from an E. coli species (Bolivar, et al., Gene 2: 95 [1977]). pBR322 contains genes for ampicillin and tetracycline resistance and thus provides easy means for identifying transformed cells, whether for purposes of cloning or expression. Expression vectors also optimally will contain sequences which are useful for the control of transcription and translation, e.g., promoters and Shine-Dalgarno sequences (for prokaryotes) or promoters and enhancers (for mammalian cells). The promoters may be, but need not be, inducible; even powerful constitutive promoters such as the CMV promoter for mammalian hosts have been found to produce the LHR without host cell toxicity. While it is conceivable that expression vectors need not contain any expression control, replicative sequences or selection genes, their absence may hamper the identification of hybrid transformants and the achievement of high level hybrid immunoglobulin expression.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “epitope tagged” when used herein refers to a chimeric polypeptide comprising a FIZZ polypeptide fused to a “tag polypeptide.” The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

The term “antibody” is used in the broadest sense and specifically covers single anti-FIZZ monoclonal antibodies (including agonist, antagonist, and neutralizing antibodies) and anti-FIZZ antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts.

As use herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and the immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of FIZZ which retain the biologic and/or immunologic activities of native or naturally-occurring FIZZ. A preferred activity is the ability of the FIZZ molecule to inhibit neurotrophin action on neurons, which can, for example, be tested by monitoring either the inhibition of neuronal survival in an embryonic rat DRG Neuronal Survival Inhibition Assay, or the inhibition of CGRP upregulation seen with a neurotrophin (e.g., NGF) in adult DRG neurons (see the Examples).

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial effect for an extended period of time.

“Mammal” for purposes of treatment refers to any animal classified as a mammal, including humans, domestic and farm animals, and zoo, sports, or pet animals, such as dogs, cats, cows, horses, sheep, pigs, etc. Preferably, the mammal is human.

Administration “in combination with” one or more further therapeutic agents includes simultaneous (concurrent) and consecutive administration in any order.

The term “antagonist” is used in the broadest sense, and includes any molecule that blocks, prevents, inhibits, or neutralizes a biological activity of a native FIZZ polypeptide. In a similar manner, the term “agonist” is used in the broadest sense and includes any molecule that mimics, or enhances a biological activity of a native FIZZ polypeptide. Suitable agonist or antagonist molecules specifically include agonist or antagonist antibodies or antibody fragments, fragments or amino acid sequence variants of native FIZZ polypeptides, peptides, small organic molecules, etc.

II. Compositions and Methods of the Invention

A. Full-Length FIZZ Polypeptides

The present invention provides newly identified and isolated polypeptides referred to in the present application as FIZZ. In particular, Applicants have identified and isolated cDNA encoding a family of murine and human FIZZ polypeptides, as disclosed in further detail in the Examples below. The first FIZZ polypeptide (m-FIZZ1) was isolated from the airways of asthmatic mice by gel electrophoresis. Although the molecular weight of this protein was found to be similar to the molecular weight of chemokines (about 8 to 9 kDa), to applicants present knowledge, the m-FIZZ1 sequence encodes a novel factor; using BLAST (such as, WU-BLAS-2) and FastA sequence alignment computer programs, no significant sequence identities to any known proteins were revealed. The other (murine and human) FIZZ proteins were generated by homology searches of EST databases, using the m-FIZZ1 sequence, and (similarly to m-FIZZ1) show no significant homology to any known proteins.

B. FIZZ Variants

In addition to the full-length native FIZZ polypeptides described herein, it is contemplated that FIZZ variants can be prepared. FIZZ variants can be prepared by introducing appropriate nucleotide changes into the FIZZ DNA, or by synthesis of the desired FIZZ polypeptide. Those skilled in the art will appreciate that amino acid changes may alter post-translational processes of the FIZZ, such as changing the number or position of glycosylation sites or altering the membrane anchoring characteristics.

Variations in the native full-length sequence FIZZ or in various domains of the FIZZ polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the FIZZ that results in a change in the amino acid sequence of the FIZZ as compared with the native sequence FIZZ. Optionally the variation is by substitution of at least one amino acid with any other amino acid in one or more of the domains of the FIZZ. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity in the in vitro assay described in the Examples below.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the FIZZ variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

C. Modifications of the FIZZ Polypeptides

Covalent modifications of FIZZ polypeptides are included within the scope of this invention. One type of covalent modification includes reacting targeted amino acid residues of a given FIZZ molecule with an organic derivatizing agent that is capable of reacting with selected side chains or the N- or C-terminal residues of the FIZZ. Derivatization with bifunctional agents is useful, for instance, for crosslinking FIZZ to a water-insoluble support matrix or surface for use in the method for purifying anti-FIZZ antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis-(succinimidylpropionate), bifunctional maleimides such as bis-N-maleimido-1,8-octane and agents such as methyl-3-[(p-azidophenyl)-dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the FIZZ polypeptide included within the scope of this invention comprises altering the native glycosylation pattern of the polypeptide. “Altering the native glycosylation pattern” is intended for purposes herein to mean deleting one or more carbohydrate moieties found in native sequence FIZZ, and/or adding one or more glycosylation sites that are not present in the native sequence FIZZ and/or chemically or enzymatically changing the extent or composition of the native glycosylation of a FIZZ polypeptide.

Addition of glycosylation sites to the FIZZ polypeptide may be accomplished by altering the amino acid sequence. The alteration may be made, for example, by the addition of, or substitution by, one or more serine or threonine residues to the native sequence FIZZ (for 0-linked glycosylation sites). The FIZZ amino acid sequence may optionally be altered through changes at the DNA level, particularly by mutating the DNA encoding the FIZZ polypeptide at preselected bases such that codons are generated that will translate into the desired amino acids.

Another means of increasing the number of carbohydrate moieties on the FIZZ polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the FIZZ polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of FIZZ comprises linking the FIZZ polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

The FIZZ polypeptides of the present invention may also be modified in a way to form a chimeric molecule comprising a FIZZ polypeptide fused to another, heterologous polypeptide or amino acid sequence. In one embodiment, such a chimeric molecule comprises a fusion of the a FIZZ with a tag polypeptide which provides an epitope to which an anti-tag antibody can selectively bind. The epitope tag is generally placed at the amino- or carboxyl-terminus of the FIZZ. The presence of such epitope-tagged forms of the FIZZ polypeptides can be detected using an antibody against the tag polypeptide. Also, provision of the epitope tag enables the FIZZ to be readily purified by affinity purification using an anti-tag antibody or another type of affinity matrix that binds to the epitope tag. In an alternative embodiment, the chimeric molecule may comprise a fusion of the FIZZ with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In another embodiment, the FIZZ polypeptides (including their fragments) or anti-FIZZ antibodies or antibody fragments are fused to toxins, such as ricin, saporin or pseudomonas endotoxin. Such fusions are used to deliver the toxins to desired tissues to which the FIZZ polypeptide or the anti-FIZZa antibody binds.

In a further embodiment, the chimeric molecule comprises a FIZZ polypeptide sequence fused to an immunoglobulin constant region sequence. The fusion is preferably to a heavy chain constant region sequence, e.g., a hinge, CH2 and CH3 regions, or the CH1, hinge, CH2 and CH3 regions of an IgG immunoglobulin. As discussed earlier, such chimeric molecules are commonly referred to as immunoadhesins.

D. Preparation of the FIZZ Polypeptides

The description below relates primarily to production of FIZZ polypeptides by culturing cells transformed or transfected with a vector containing nucleic acid encoding the desired FIZZ. It is, of course, contemplated that alternative methods, which are well known in the art, may be employed to prepare FIZZ polypeptides. For instance, the FIZZ sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the FIZZ protein may be chemically synthesized separately and combined using chemical or enzymatic methods to produce the full-length FIZZ.

1. Isolation of DNA Encoding FIZZ Polypeptides

DNA encoding FIZZ polypeptides may be obtained from a cDNA library prepared from tissue believed to possess the FIZZ mRNA and to express it at a detectable level. For example, murine FIZZ DNA can be obtained from a cDNA library prepared from the lungs of asthmatic mice. Human FIZZ DNA can be conveniently obtained from a cDNA library prepared from human tissue, such as described in the Examples. The FIZZ-encoding gene may also be obtained from a genomic library or by oligonucleotide synthesis.

Libraries can be screened with probes (such as antibodies to the FIZZ or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding FIZZ is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs such as ALIGN, DNAstar, and INHERIT which employ various algorithms to measure homology.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for FIZZ production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for FIZZ-encoding vectors. Saccharomyces cerevisiae is a commonly used lower eukaryotic host microorganism.

Suitable host cells for the expression of glycosylated FIZZ polypeptides are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding a FIZZ polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The FIZZ polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the FIZZ DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the FIZZ-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the FIZZ nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S. D.) sequence operably linked to the DNA encoding FIZZ.

Examples of suitable promoting sequences for use with yeast hosts include the promoters for 3-phosphoglycerate kinase (Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

FIZZ transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the FIZZ polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the FIZZ coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding FIZZ.

Still other methods, vectors, and host cells suitable for adaptation to the synthesis of FIZZ polypeptides in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal or bird. Conveniently, the antibodies may be prepared against a native sequence FIZZ polypeptide or against a synthetic peptide based on the DNA sequences provided herein or against exogenous sequence fused to FIZZ DNA and encoding a specific antibody epitope.

5. Purification of FIZZ Polypeptides

Forms of FIZZ polypeptides may be recovered from culture medium or from host cell lysates. If membrane-bound, it can be released from the membrane using a suitable detergent solution (e.g. Triton-X 100) or by enzymatic cleavage. Cells employed in expression of FIZZ can be disrupted by various physical or chemical means, such as freeze-thaw cycling, sonication, mechanical disruption, or cell lysing agents.

It may be desired to purify the FIZZ polypeptides from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the FIZZ. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular FIZZ produced.

E. Uses for the FIZZ Polypeptides

The FIZZ polypeptides disclosed herein can be used in assays to identify their receptor(s) and/or other factors mediating their biological actions. In addition, by such methods, inhibitors of the interaction of FIZZ polypeptides with their receptor(s) can be identified. The FIZZ proteins can also be used to screen for peptide or small molecule inhibitors or agonists of FIZZ biological activity (e.g., neurotrophin inhibitory activity). A specific FIZZ can also be employed to isolate any native molecule specifically binding to it. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

In vitro assays employ a mixture of components including a FIZZ polypeptide, which may be part of fusion product with another peptide or polypeptide, e.g., a tag for detecting or anchoring, etc. The assay mixtures may further comprise one or more neurotrophin, responsive neurons and/or (for binding assays) a natural intra- or extracellular FIZZ binding target. While native binding targets may be used, it is frequently preferred to use a portion of such native binding targets (e.g. peptides), so long as the portion provides binding affinity and avidity to the subject FIZZ protein conveniently measurable in the assay. The assay mixture also contains a candidate pharmacological agent. Candidate agents encompass numerous chemical classes, though typically they are organic compounds, preferably small organic compounds, and are obtained from a wide variety of sources, including libraries of synthetic or natural compounds. A variety of other reagents may also be included in the mixture, such as, salts, buffers, neutral proteins, e.g. albumin, detergents, protease inhibitors, nuclease inhibitors, antimicrobial agents, etc.

In in vitro binding assays, the resultant mixture is incubated under conditions whereby, but for the presence of the candidate molecule, the FIZZ protein specifically binds the cellular binding target, portion or analog, with a reference binding affinity. The mixture components can be added in any order that provides for the requisite bindings and incubations may be performed at any temperature which facilitates optimal binding. Incubation periods are likewise selected for optimal binding but also minimized to facilitate rapid high-throughput screening.

After incubation, the agent-biased binding between the FIZZ protein and one or more binding targets is detected by any convenient technique. For cell-free binding type assays, a separation step is often used to separate bound from unbound components. Separation may be effected by precipitation (e.g. TCA precipitation, immunoprecipitation, etc.), immobilization (e.g on a solid substrate), etc., followed by washing by, for example, membrane filtration (e.g. Whatman's P-18 ion exchange-paper, Polyfiltronic's hydrophobic GFC membrane, etc.), gel chromatography (e.g. gel filtration, affinity, etc.). For FIZZ-dependent transcription assays, binding is detected by a change in the expression of a FIZZ-dependent reporter.

Detection may be effected in any convenient way. For cell-free binding assays, one of the components usually comprises or is coupled to a label. The label may provide for direct detection as radioactivity, luminescence, optical or electron density, etc., or indirect detection, such as, an epitope tag, an enzyme, etc. A variety of methods may be used to detect the label depending on the nature of the label and other assay components, e.g. through optical or electron density, radiative emissions, nonradiative energy transfers, etc. or indirectly detected with antibody conjugates, etc.

In a preferred embodiment, the ability of a FIZZ polypeptide, antagonist, agonist or anti-FIZZ antibody to modify neurotrophin action is tested in a kinase receptor activation (KIRA) assay, in an enzyme-linked immunosorbent (ELISA) assay format, as described, for example, in PCT publication WO 95/14930 published Jun. 1, 1995. The KIRA assay measures the activation (autophosphorylation) of a tyrosine kinase receptor of interest. The assay can be divided into two major stages, each of which is generally performed in separate assay plates. The first stage of the assay involves activating the receptor and is termed the kinase receptor activation (KIRA) stage of the assay. The second stage involves measuring receptor activation. Conveniently, this is achieved using an enzyme-linked immunosorbent assay (ELISA) to measure receptor activation. In general, the KIRA ELISA assay involves the following steps: (a) coating a first solid phase with a homogeneous population of eukaryotic cells so that the cells adhere to the first solid phase, wherein, positioned in their membrane, the cells have a receptor construct comprising a flag polypeptide and the tyrosine kinase receptor; (b) exposing the adhering cells to an analyte; (c) solubilizing the adhering cells, thereby releasing cell lysate; (d) coating a second solid phase with a capture agent which binds specifically to the flag polypeptide so that the capture agent adheres to the second solid phase; (e) exposing the adhering capture agent to the cell lysate obtained in step (c) so that the receptor construct adheres to the second solid phase; (f) washing the second solid phase so as to remove unbound cell lysate; (g) exposing the adhering receptor construct to an anti-phosphotyrosine antibody which identifies phosphorylated tyrosine residues in the tyrosine kinase receptor; and (h) measuring binding of the anti-phosphotyrosine antibody to the adhering receptor construct.

Further details of various screening approaches to identify small-molecule lead compounds are disclosed, for example, in Bevan et al., Tibtech 13, 115-121 (1995), and Hodgson, Bio/Technology 11, 683-688 (1993).

The FIZZ polypeptide may also be useful in the diagnosis or treatment (including prevention) of various pathological states characterized by altered nerve function, such as, neuropathy, ALS, impotence, hypertension, chronic pain, asthma, cystitis, bowel disease, cardiac arrhythmias, sudden cardiac death, CNS degenerative disease, wound healing, stroke, head trauma, vasogenic edema, or encephalitis. It may be possible to diagnose any of these conditions by detecting an abnormal (decreased or increased) expression of a native FIZZ protein. Treatment of these and similar conditions may, in turn, be effected by administering an effective amount of a FIZZ polypeptide, or FIZZ agonist or antagonist, as the case may be.

FIZZ proteins, agonists, antagonists, or anti-FIZZ antibodies, may also be useful in blocking the side-effects of neurotrophins.

In addition, FIZZ1 has been shown to be associated with immune-mediated inflammation, and showed immunomodulatory properties using a mixed lymphocyte reaction (MLR) assay. The MLR assay evaluates the ability of T lymphocytes to proliferate in response to the presentation of an allo-antigen. The assay identified molecules which either enhance or inhibit the proliferation of the responder T lymphocyte in response to stimulation with presented allo-antigen. m-FIZZ and h-FIZZ proteins, tested at various concentrations, have been found to stimulate MLR response. Accordingly, these molecules (and small molecule or antibody agonists of the receptor(s) for these molecules) are promising therapeutic agents in situations where enhancement of immune response would be beneficial. Accordingly, the FIZZ proteins (and their agonists) may be utilized to enhance the immune response to infectious agents, and could, therefore, find utility in the treatment of infectious diseases, such as, HIV infection, hepatitis A, B, C, D, E infection, bacterial infections, fungal infections, protozoa and parasitic infections, etc. In addition, the FIZZ proteins and other molecule that similarly stimulate the MLR, may be used to enhance the immune response for conditions of inherited acquired, infection induced (e.g. HIV), or ioatrogenic (e.g. as from chemotherapy) immunodeficiency. For FIZZ1, the MLR results suggest that this protein (and its agonists) may function to enhance the mucosal immune response in the lung.

It has been demonstrated that some human cancer patients develop an antibody or T lymphocyte response to antigens on neoplastic cells. It has also been shown in animal models or neoplasia that enhancement of the immune response can result in rejection or regression of that particular neoplasm. Molecules that enhance the T lymphocyte response in the MLR assay, such as, FIZZ1, may be used in vivo to enhance the immune response against neoplasia. Accordingly, such molecules (including small molecule agonists of FIZZ and antibodies that effect the same receptor in an agonist fashion), are candidates for tumor (cancer) therapy.

Nucleotide sequences (or their complement) encoding FIZZ polypeptides have various applications in the art of molecular biology, including uses as hybridization probes, in chromosome and gene mapping and in the generation of anti-sense RNA and DNA. FIZZ nucleic acid will also be useful for the preparation of FIZZ polypeptides by the recombinant techniques described herein.

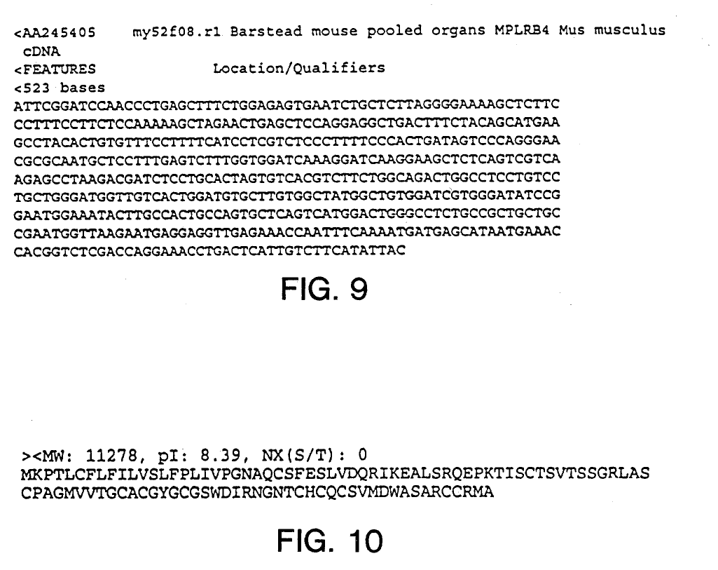

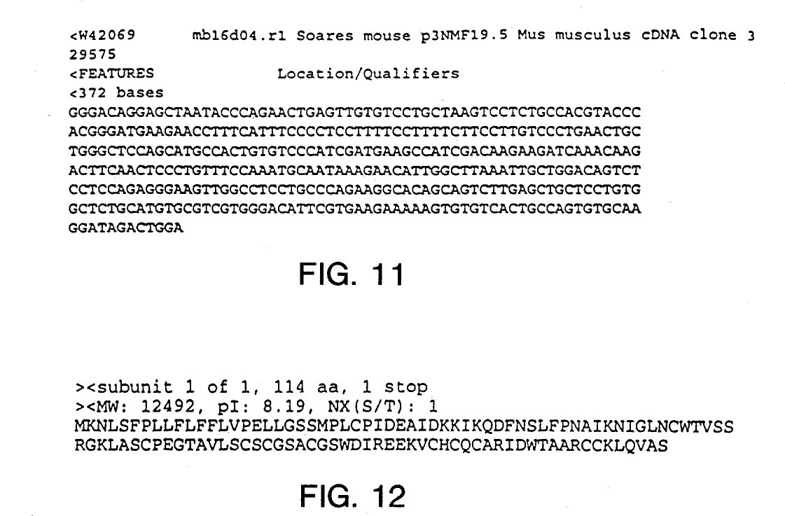

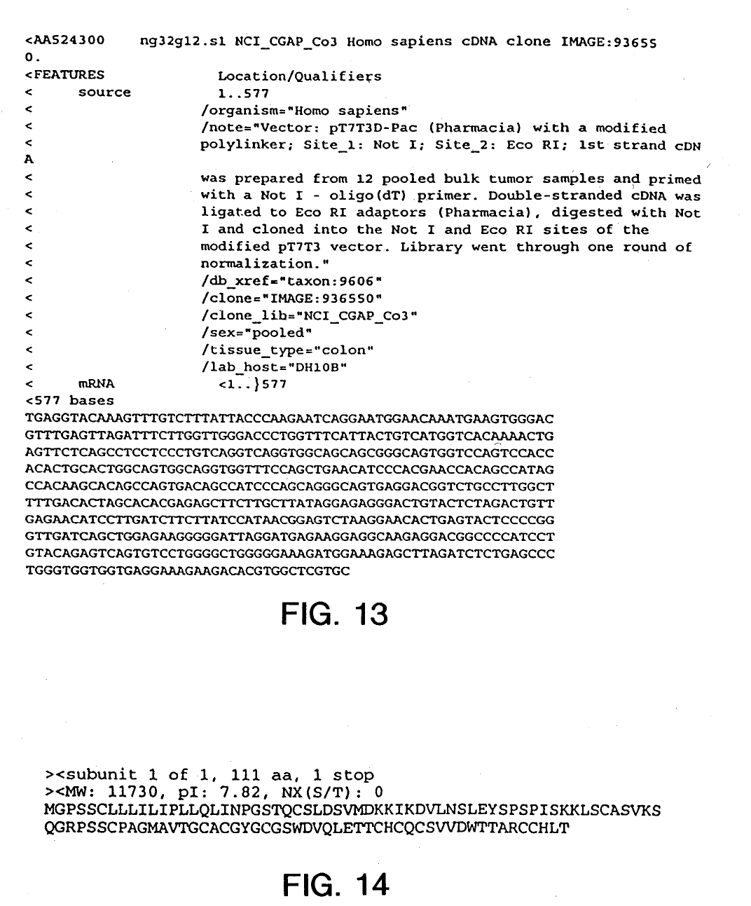

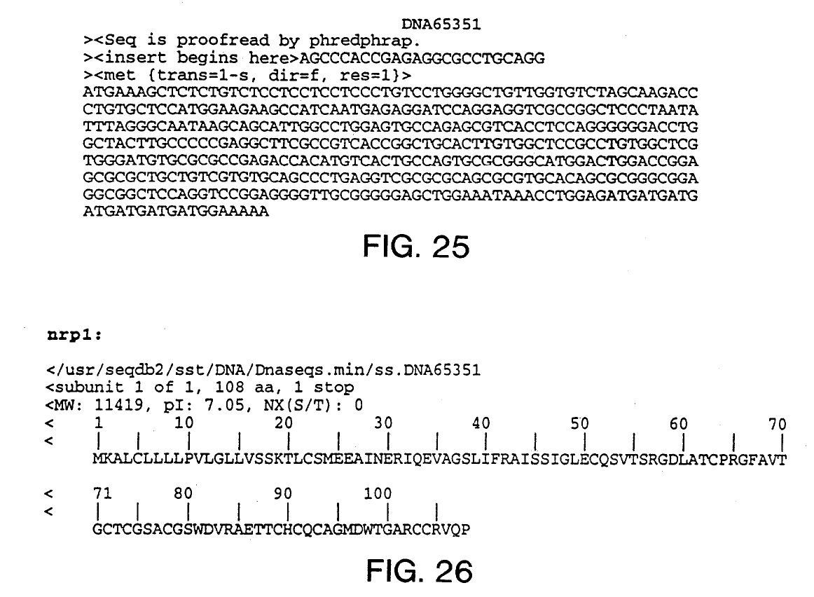

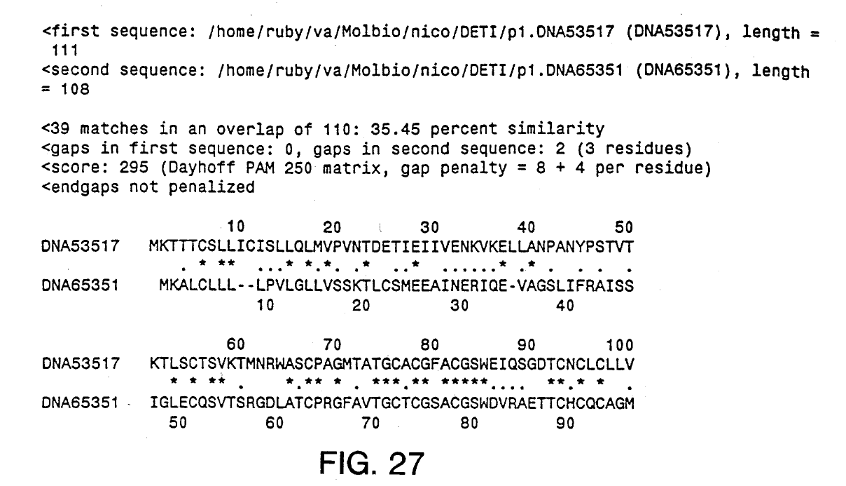

The full-length native sequence FIZZ genes, such as, m-FIZZ1 (DNA 53517, FIG. 5, SEQ ID NO: 9); m-FIZZ2 (DNA 54229, FIG. 9, SEQ ID NO: 13), m-FIZZ3 (DNA 54231, FIG. 11, SEQ ID NO: 15); h-FIZZ1 (DNA 54228, FIG. 13, SEQ ID NO: 17); hFIZZ-3 (DNA65351, FIG. 25, SEQ ID NO: 23), or portions thereof, may be used as hybridization probes for a cDNA library to isolate the full-length FIZZ gene or to isolate still other genes (for instance, those encoding naturally-occurring variants of FIZZ or FIZZ polypeptides from other species) which have a desired sequence identity to any of the murine or human FIZZ sequences specifically disclosed herein. Optionally, the length of the probes will be about 20 to about 50 bases. The hybridization probes may be derived from the nucleotide sequences of SEQ ID NOs: 9, 13, 15, 17, or 19, or from genomic sequences including promoters, enhancer elements and introns of native sequence FIZZ polypeptides. By way of example, a screening method will comprise isolating the coding region of a native FIZZ gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of a FIZZ gene of the present invention can be used to screen libraries of human cDNA, genomic DNA or mRNA to determine which members of such libraries the probe hybridizes to. Hybridization techniques are described in further detail in the Examples below.

The probes may also be employed in PCR techniques to generate a pool of sequences for identification of closely related FIZZ sequences.

Nucleotide sequences encoding a FIZZ polypeptide can also be used to construct hybridization probes for mapping the gene which encodes that FIZZ polypeptide and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

Nucleic acids which encode FIZZ polypeptides or its modified forms can also be used to generate either transgenic animals or “knock out” animals which, in turn, are useful in the development and-screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding a FIZZ polypeptide can be used to clone genomic DNA encoding that FIZZ in accordance with established techniques and the genomic sequences used to generate transgenic animals that contain cells which express DNA encoding the FIZZ. Methods for generating transgenic animals, particularly animals such as mice or rats, have become conventional in the art and are described, for example, in U.S. Pat. Nos. 4,736,866 and 4,870,009. Typically, particular cells would be targeted for FIZZ transgene incorporation with tissue-specific enhancers. Transgenic animals that include a copy of a transgene encoding FIZZ introduced into the germ line of the animal at an embryonic stage can be used to examine the effect of increased expression of DNA encoding FIZZ. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of a FIZZ polypeptide can be used to construct a FIZZ “knock out” animal which has a defective or altered gene encoding FIZZ as a result of homologous recombination between the endogenous gene encoding the FIZZ and altered genomic DNA encoding FIZZ introduced into an embryonic cell of the animal. For example, cDNA encoding FIZZ can be used to clone genomic DNA encoding FIZZ in accordance with established techniques. A portion of the genomic DNA encoding FIZZ can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the FIZZ polypeptide.

Nucleic acid encoding FIZZ polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically. retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of the currently known gene marking and gene therapy protocols see Anderson et al., Science 256, 808-813 (1992).

F. Anti-FIZZ Antibodies

The present invention further provides anti-FIZZ antibodies. Exemplary antibodies include polyclonal, monoclonal, humanized, bispecific, and heteroconjugate antibodies.

1. Polyclonal Antibodies

The anti-FIZZ antibodies may comprise polyclonal antibodies. Methods of preparing polyclonal antibodies are known to the skilled artisan. Polyclonal antibodies can be raised in a mammal or bird, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the FIZZ polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-FIZZ antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include a FIZZ polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the FIZZ. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Humanized and Human Antibodies

The anti-FIZZ antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

4. Bispecific Antibodies

Bispecific antibodies are monoclonal, preferably human or humanized, antibodies that have binding specificities for at least two different antigens. In the present case, one of the binding specificities is for a FIZZ polypeptide, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

G. Uses for Anti-FIZZ Antibodies

The anti-FIZZ antibodies of the invention have various utilities. For example, anti-FIZZ antibodies may be used in diagnostic assays for FIZZ, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-FIZZ antibodies also are useful for the affinity purification of FIZZ polypeptides from recombinant cell culture or natural sources. In this process, the antibodies against a particular FIZZ are immobilized on a suitable support, such a Sephadex resin or filter paper, using methods well known in the art. The immobilized antibody then is contacted with a sample containing the FIZZ to be purified, and thereafter the support is washed with a suitable solvent that will remove substantially all the material in the sample except the FIZZ, which is bound to the immobilized antibody. Finally, the support is washed with another suitable solvent that will release the FIZZ from the antibody.

Agonist or antagonist-anti-FIZZ antibodies may be useful in the diagnosis or treatment (including prevention) of diseases, conditions or pathological states characterized by altered nerve function, such as, neuropathy, ALS, impotence, hypertension, chronic pain, asthma, cystitis, bowel disease, cardiac arrhythmias, sudden cardiac death, or CNS degenerative diseases It may be possible to diagnose these conditions by detecting an abnormal (decreased or increased) expression of a native FIZZ protein by using an anti-FIZZ antibody specifically binding to the targeted FIZZ molecule. Treatment of these and similar conditions may, in turn, be effected by administering an effective amount of an agonist or antagonist anti-FIZZ antibody, as the case may be, in an effective amount.

Anti-FIZZ1 antibodies specifically could be useful in the treatment of inflammatory and fibrotic lung diseases, e.g., eosinophilic pneumonias, idiopathic pulmonary fibrosis, and hypersensitivity pneumonitis. These diseases may involve a disregulated immune-inflammatory response, the inhibition of which would be of therapeutic benefit. Antibodies to the FIZZ proteins, and in particular FIZZ1, may also be useful in the treatment of inflammatory bowel disease (IBD, including ulcerative colitis and Crohn's disease), gluten-sensitive enteropathy, or Whipple's disease.

H. Pharmaceutical Compositions

For therapeutic uses, the FIZZ polypeptides, their agonist or antagonists, including, without limitation, anti-FIZZ antibodies, are administered in the form of a pharmaceutical composition comprising one or more of these molecules as an active ingredient, in conjunction with a pharmaceutically acceptable carrier. Therapeutic formulations are prepared for storage by mixing the active ingredient(s) having the desired degree of purity with optional physiologically acceptable carriers, excipients, or stabilizers (Remington's Pharmaceutical Sciences, supra), in the form of lyophilized cake or aqueous solutions. Acceptable carriers, excipients, or stabilizers are non-toxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics, or polyethylene glycol (PEG).

The FIZZ polypeptides, or their agonists or antagonists, including anti-FIZZ antibodies, to be used for in vivo administration must be sterile. This is readily accomplished by filtration through sterile filtration membranes, prior to or following lyophilization and reconstitution. The FIZZ polypeptides ordinarily will be stored in lyophilized form or in solution.

The therapeutically effective dose will, of course, vary depending on the actual active ingredient, and on such factors as the pathological condition to be treated (including prevention), the patient's age, weight, general medical condition, medical history, etc., and its determination is well within the skill of a practicing physician. The effective dose generally is within the range of from about 0.001 to about 1.0 mg/kg, more preferably about 0.01-1 mg/kg, most preferably about 0.01-0.1 mg/kg.

It should be appreciated that endotoxin contamination should be kept minimally at a safe level, for example, less than 0.5 ng/mg protein. Moreover, for human administration, the liquid formulations should meet sterility, pyrogenicity, general safety, and purity as required by FDA Office and Biologics standards.

The route of administration is in accord with known methods, e.g., injection or infusion by intravenous, intraperitoneal, intracerebral, intramuscular, intraocular, intraarterial, or intralesional routes, or by sustained-release systems as noted below. Therapeutic compositions generally are placed into a container having a sterile access port, for example, an intravenous solution bag or vial having a stopper pierceable by a hypodermic injection needle. The formulations are preferably administered as repeated intravenous (i.v.), subcutaneous (s.c.) or intramuscular (i.m.) injections, or as aerosol formulations suitable for intranasal or intrapulmonary delivery (for intrapulmonary delivery see, e.g. EP 257,956).

The FIZZ polypeptides, their agonists or antagonists, can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 [1981] and Langer, Chem. Tech., 12: 98-105 [1982] or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 [1983]), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release FIZZ, FIZZ agonist or FIZZ antagonist compositions also include liposomally entrapped active ingredients. Liposomes containing such active ingredients are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The FIZZ polypeptides, their agonists or antagonists, may be administered in combination with other therapeutic agents used for the treatment of pathological conditions associated with altered neurotrophin function. Preferred candidates for combination therapy are neurotrophic factors (as hereinbefore described), or their agonists or antagonists, including antibodies specifically binding and blocking or mimicking a biological activity of a native neutrotrophin.

The effective amount of the therapeutic agents administered in combination with the FIZZ polypeptides, agonists or antagonists herein, will be at the physician's or veterinarian's discretion. Dosage administration and adjustment is done to achieve optimal management of the conditions to be treated, and ideally takes into account use of diuretics or digitalis, and conditions such as hyper- or hypotension, renal impairment, etc. The dose will additionally depend on such factors as the type of the therapeutic agent to be used and the specific patient being treated. Typically, the amount employed will be the same dose as that used, if the given therapeutic agent is administered without the FIZZ polypeptides, agonists or antagonists herein.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, 10801 University Blvd., Manassas, Va. 20110-2209 (ATCC).

### Example 1

Identification and Cloning of m-FIZZ1 (DNA53517)

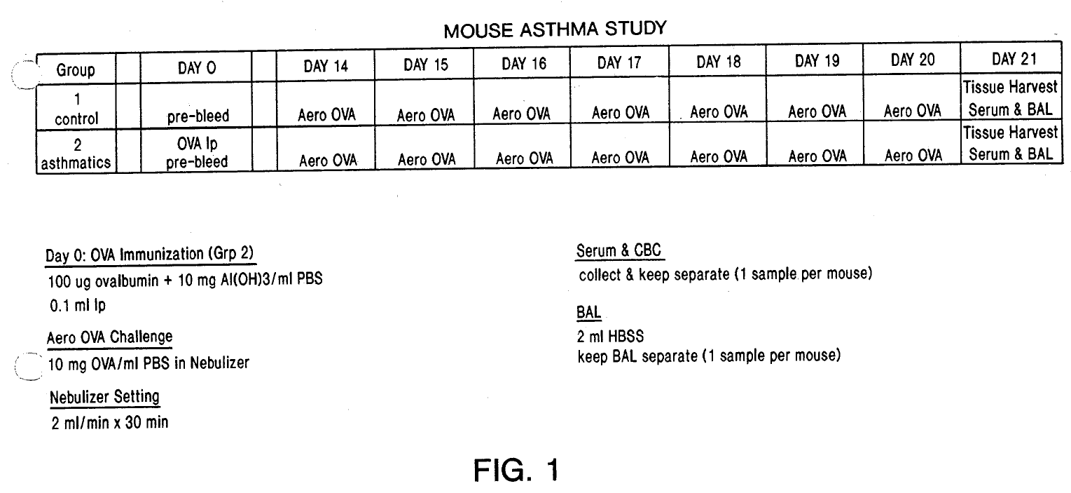

Mouse asthma model Female Balb/C mice, 6 to 8 weeks of age, were separated into two experimental groups: controls and asthmatics. The asthmatic group was immunized intraperitoneally with 10 μg ovalbumin+1 mg alum, while the control group was not. Two weeks later, mice were exposed daily to an aerosol of 10 mg/ml ovalbumin in PBS aerosolized with a UltraNeb nebulizer (DeVilbiss) at the rate of 2 ml/min for 30 min each day, for 7 consecutive days. One day after the last aerosol challenge, whole blood, serum and bronchoalveolar lavage (BAL) samples were collected and the lungs were harvested and preserved for histological examination, immuno-histochemistry and in situ hybridization. A schematic protocol is shown in FIG. 1.

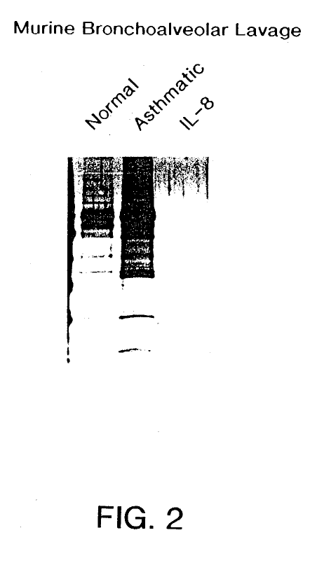

Gel electrophoresis of BAL samples Examination of the BAL samples by gel electrophoresis on a 16% Tricine gel shows that a low molecular weight protein is expressed in the BAL samples from asthmatic mice but not in the BAL samples from control mice. This low molecular weight protein, which we named m-DET1 (referring to the first three N-terminal amino acids), and then renamed m-FIZZ1, co-migrates with a 8300 Dalton marker protein (IL-8) (FIG. 2).

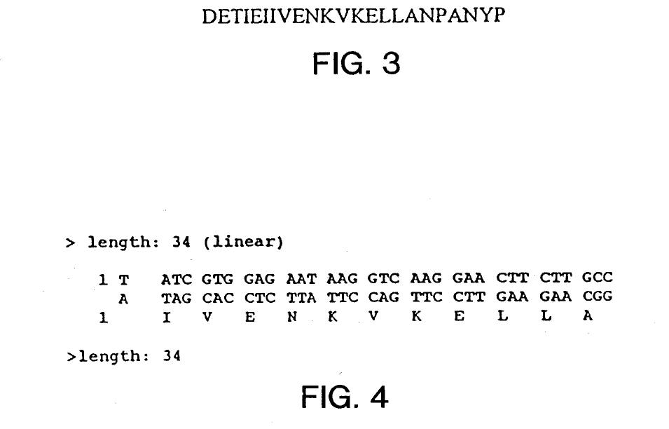

Partial protein sequence The protein of interest was transferred upon a PVDF membrane and sequenced by Edman degradation. The first 23 amino acids of the N-terminal sequence are shown in FIG. 3 (SEQ ID NO: 1).

Partial cDNA sequence We designed two degenerate oligonucleotide PCR primers corresponding to the putative DNA sequence for the first 7 and the last 7 amino acids in our sequence of 23, respectively.

Oligo #1: coding for DETIEI, with MluI overhang ACA AAC GCG TGA YGA RAC NAT HGA RAT (SEQ ID NO: 2)

Oligo #2: coding for NPANYP, with SphI overhang TGG TGC ATG CGG RTA RTT NGC NGG RTT (SEQ ID NO: 3)

cDNA prepared from the lungs of normal mice was used as a template for the PCR reaction which yielded an 88 bp product. This 88 bp product contained 54 known base pairs, encoding the PCR primers, and 34 novel base pairs, encoding the intervening amino acids in the FIZZ sequence, as shown in FIG. 4. The strands of the double-stranded nucleic acid molecule shown in FIG. 4 are identified as SEQ ID NOs 4 and 5, respectively, while the encoded amino acid sequence is designated as SEQ ID NO: 6.

Full length cDNA clone The partial sequence was used to design primers which were used to obtain a full length FIZZ clone by RT-PCR of mouse lung poly(A)⁺ RNA

Oligo #3:

ACA AAC GCG TGC TGG AGA ATA AGG TCA AGG (SEQ ID NO: 7)

This oligo was used as an RT-PCR primer in combination with 5′ and 3′ amplimers from Clontech.

Oligo #4:

ACT AAC GCG TAG GCT AAG GAA CTT CTT GCC (SEQ ID NO: 8)

This oligo was used as an RT-PCR primer in combination with oligo d(T).

The complete m-FIZZ1 cDNA and protein sequences are shown in FIG. 5. The coding strand of the full length cDNA is also included as SEQ ID NO: 9, while the full length deduced protein sequence is designated as SEQ ID NO: 10, and includes a putative signal sequence of 23 amino acids, the mature N-terminus starting a position 24, with the sequence motif DET.

### Example 2

Expression and Purification of m-FIZZ1

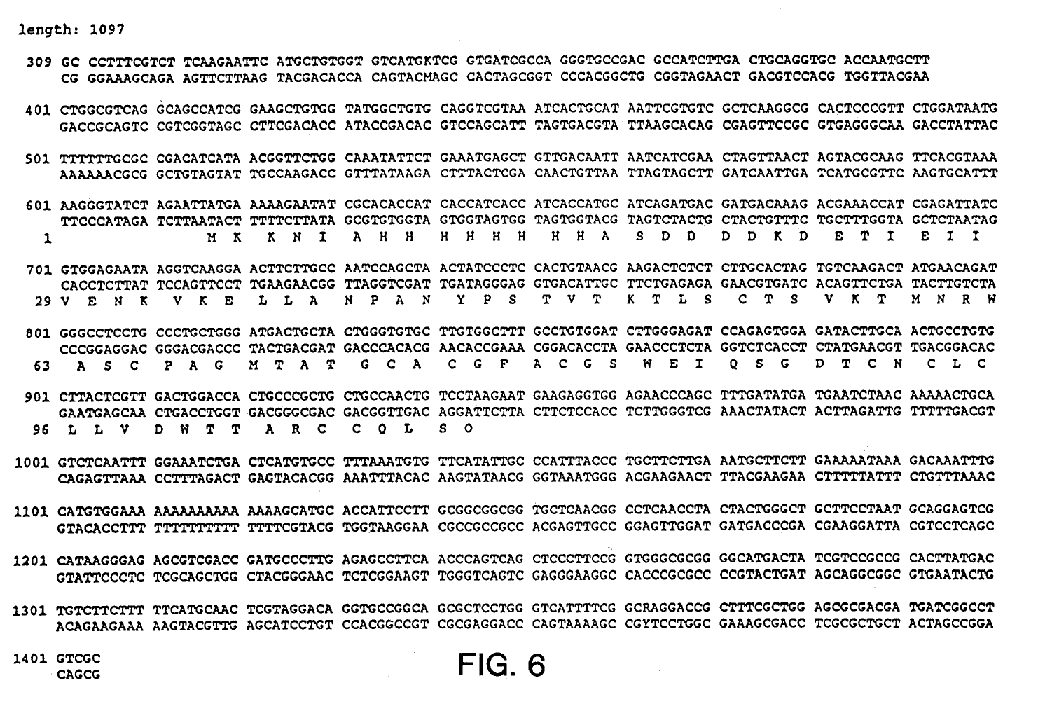

Construction of an expression vector A FIZZ1 expression vector (designated pST31-FIZZ1) was constructed using the pST31 E. coli expression plasmid which contains a trp promoter, a portion of an ST2 sequence, a poly(His) tag and an enterokinase cleavage site. Briefly, we used the NsiI and SpH″I sites of pST31 to subclone FIZZ cDNA lacking the signal sequence, using a short linker to cover the bases corresponding to the enterokinase cleavage sequence, located between the NsiI site and the 5′ end of the FIZZ clone as shown in FIG. 6.

Expression in E. coli pST31-FIZZ1 was used to transformed a protease deficient mutant of E. coli strain W3110, using the methods described in Sambrook et al., Molecular Cloning: A Laboratory Manual (New York: Cold Spring Harbor Laboratory Press, 1989). Transformants were identified by their ability to grow on LB plates and antibiotic resistant colonies were then selected.

Extraction of E. coli Produced Protein: 25 gms of E. coli paste #DRS-307a was added to 500 mls of 0.1M Tris, 7M guanidine (pH 9.). The sample was mixed until completely dissolved. Upon dissolving, sodium sulfite and sodium tetrathionate were added to a final concentration of 0.1 M and 20 mM, respectively, and the sample was continually stirred for one hour at room temperature. Following incubation, the sample was spun at 45,000 rpm for 30 minutes in a Beckman ultracentrifuge. The supernatant was filtered through a 0.45 micron filter and loaded onto a Ni—NTA column. The column was washed with 20 mM glycine, 300 mM NaCl, 6.0 M urea, pH 7.5. The sample was eluted from the column with the above buffer, containing 250 mM imidazole.

Refolding of Sample Eluted from the Ni—NTA Column: A refolding buffer, containing 20 mM glycine, 300 mM NaCl, 5 mM EDTA, 4M urea, and 0.4M arginine was prepared. The pH was adjusted to 9.0, and 5 mM cysteine was added. Ni—NTA column was added to the refolding buffer, and elution was performed at a concentration of 100 μg/ml. After incubation overnight at 4° C., the refolding buffer sample was loaded onto a 100×4.6 mm C4 column, and the various refold species were separated using a 25% to 48% acetonitrile gradient. Fractions of interest were identified by electrophoresis on a 15% Tricine gel. Fractions were dialyzed against 30 mM acetate, and 150 mM NaCl (pH 4.5).

### Example 3

Northern Blot Analysis

Expression of m-FIZZ1 mRNA in various mouse tissues was examined by Northern blot analysis. Murine RNA blots were hybridized to the following ³²P-labelled DNA probe based on the full length m-FIZZ1 cDNA:

ATC TGT TCA TAG TCT TGA CAC TAG TGC AAG AGA GAG TCT TCG TTA CAG TG (SEQ ID NO: 11)

Mouse Multiple Tissue (Clontech) was incubated with the DNA probe. Blots were incubated with the probe in hybridization buffer (5×SSPE; 2× Denhardt's solution; 100 mg/mL denatured sheared salmon sperm DNA; 50% formamide; 2% SDS) for 60 hours at 42° C. The blots were washed several times in 2×SSC; 0.05% SDS for 1 hour at room temperature, followed by a 30 minute wash in 0.1×SSC; 0.1% SDS at 50° C. The blots were developed after overnight exposure by phosphorimager analysis (Fuji).

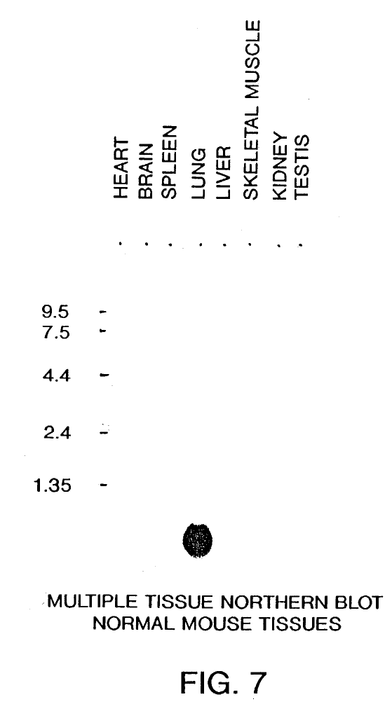

As shown in FIG. 7, results of the Northern Blot show strong expression of FIZZ1 mRNA in lung tissue, but mRNA is also detectable in cardiac tissue and skeletal muscle, although to a lesser degree (FIG. 7).

### Example 4

In Situ Hybridization

Normal and inflamed mouse lungs, normal stomach, bowel, colon tissues and bowel tissues from an experimental model of inflammatory bowel disease (IBD) were sectioned and processed for in situ hybridization my a modification of the method described by Lu and Gillett, Cell Vision 1: 169-176 (1994). [³³P]UTP-labeled sense and antisense riboprobes were generated from PCR products synthesized with T3 and T7 promoters at opposite ends. The riboprobes spanned from nucleotide 1 to nucleotide 377 of the m-FIZZ1 sequence.

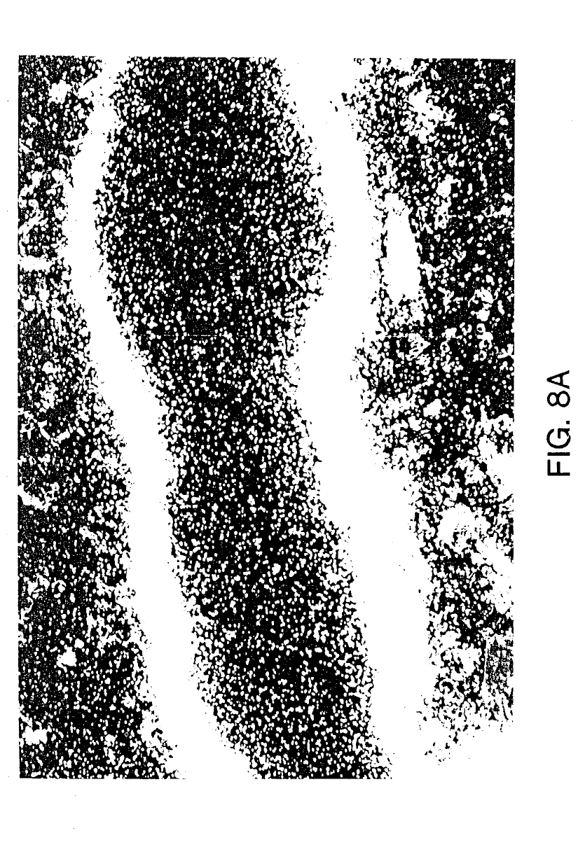

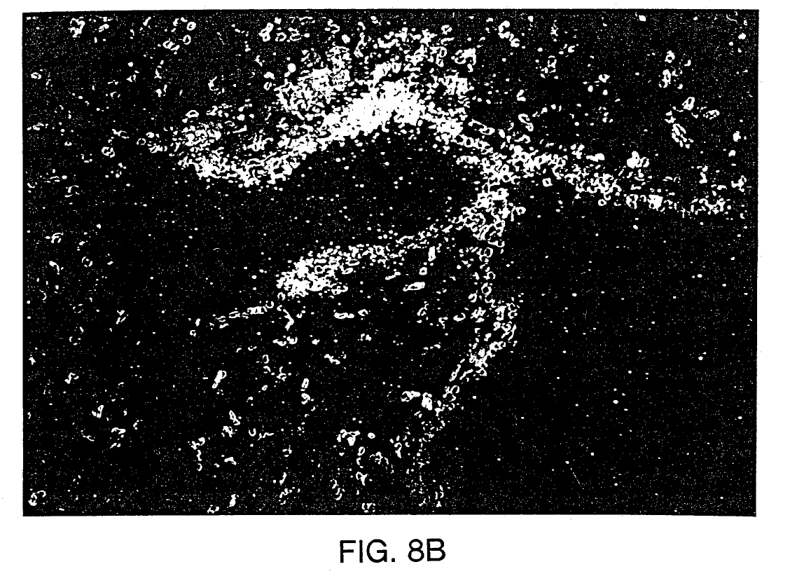

(a) m-FIZZ1 expression was examined in 36 tissue samples in two experiments. In normal lung, there was specific but multifocal or patchy expression in the mucosal epithelium of large airways (bronchi and bronchioles). In inflamed lung (procured from an asthma model) the expression in large airway mucosal epithelium was diffuse and the intensity of the signal markedly increased compared to normal lung. The results of in situ hybridization of tissue sections from asthmatic and control mouse lung are shown in FIG. 8. In inflamed lung, there was also specific expression in scattered cells closely associated with the alveolar wall. The distribution and morphology or these latter cells is most consistent with Type II alveolar pneumocytes. No signal was detected in alveolar lumenal macrophages. In stomach, bowel, colon and IBD experimental bowel, there was specific signal in scattered few interstitial cells present in the submucosa and tunica muscularis and serosa; these cells were often in the adventitia of vessel/nerve bundles. No specific signal was observed in spleen, heart, kidney, testis, or brain.

(b) In a different experiment, in situ hybridization was performed using the following probe specific for m-FIZZ1:

(SEQ ID NO: 28)

CCCAGGATGCCAACTTTGAATAGGATGAAGACTACAACTTGTTCCCTTCT
CATCTGCATCTCCCTGCTCCAGCTGATGGTCCCAGTGAATACTGATGAGA
CCATAGAGATTATCGTGGAGAATAAGGTCAAGGAACTTCTTGCCAATCCA
GCTAACTATCCCTCCACTGTAACGAAGACTCTCTCTTGCACTAGTGTCAA
GACTATGAACAGATGGGCCTCCT

The results were as follows:

Normal adult murine lung: There is patchy expression in the large airway (bronchi/bronchioles) epithelial cells. Expression is within a subset of mucosal epithelial cells. There is also expression, at an apparently equivalent level, present wihin rare discrete cells in the submucosal interstitium adjacent to the large airways. These cells, typically 1-3 within a positive focus, are adjacent to large vessels and may represent smooth muscle cells, peripheral nerves or schwann cells, of lymphatics.

Murine adult lung with allergic inflammation (eosinophilic, lymphocytic vasculitis, bronchiolitis and pneumonitis): There is diffuse string expression in all mucosal epithelial cells of all of the large airways (bronchi/bronchioles) of the lung. There is also strong expression in discrete cells that represent a subset of epithelial cells that line the alveoli; these cells are type II pneumocytes. There is also expression, as in normal lung, present within rare discrete cells in the submucosal interstitium adjacent to the large airways. These cells, typically 1 or a few cells within a positive focus, are adjacent to large vessels and may represent smooth muscle cells, peripheral nerves or schwann cells.

Normal adult murine small and large intestine: There is strong expression within multifocal few discrete single cells that are present in the submucosa, the tunica muscularis and the mesentry. The cells that express the signal are almost always associated with nerve, vein, artery triads within these areas. These cells are spindle shaped and may be either peripheral nerves, schwann cells associated with such nerves or some type of support cells associated with vessel or lymphatics. Interestingly, there is no expression within identifiable myenteric plexi that are present within the tunic muscularis.

Inflamed adult murine (IL10R KO) large intestine: In inflamed large bowel (from an IL 10R KO mouse) the pattern of expression is similar but the expression level is significantly decrease; this may be artifactual, may be a correlation with inflammation or may be a correlation with lack of IL10 signaling. Follow up work is needed to delineate these possibilities.

Murine day 12 and day 15 embryos: There is no specific expression of m-FIZZ1.

Inflamed and normal murine foot pad: There is no specific expression of m-FIZZ1.

(c) In situ hybridization was performed using the following probe specific for m-FIZZ2:

(SEQ ID NO: 29)

CCCTGAGCTTTCTGGAGAGTGAATCTGCTCTTAGGGAAAAGCTCTTCCCT
TTCCTTCTCCAAAAAGCTAGAACTGAGCTCCAGGAGGCTGACTTTCTACA
GCATGAAGCCTACACTGTGTTTCCTTTTCATCCTCGTCTCCCTTTTCCCA
CTGATAGTCCCAGGGAACGCGCAATGCTCCTTTGAGTCTTTGGTGGATCA
AAGGATGAAGGAAGCTCTCAGTCGTCAAGAGCCTAAGACGATCTCCTGCA
C

The tissue expression results were as follows:

Normal adult large intestine: There is strong segmental expression in the mucosal crypt epithelial cells; this expression is present only in crypt cells and extends approximately half way up the villi. The pithelial cells on the ends of the villi do not have signal. The pattern correlates with mucosal epithelial cell population that is capable of division. The fact that the pattern is segmental, i.e. there are some regions of large intestine with no signal, is interesting. Similarly, it is interesting that the signal is only present in the epithelial cells capable of division/proliferation.

Inflamed (IL10R KO mice) adult murine large intestine: The pattern and intensity of expression appears similar to that described above for normal large intestine.

Normal adult small intestine: There are a few segmental areas which have expression in the mucosal crypt epithelial cells; this expression is much weaker than that in the large intestine and is in only a small proportion of the small bowel. Further evaluation of the small and large intestines is warranted.

Normal and inflamed adult murine lung: No signal.

(d) In situ hybridization was also performed using the following probe specific for m-FIZZ3:

(SEQ ID NO: 30)

CGAGGGGGACAGGAGCTAATACCCAGAACTGAGTTGTGTCCTGCTAAGTC
CTCTGCCACGTACCCACGGGATGAAGAACCTTTCATTTCCCCTCCTTTTC
CTTTTCTTCCTTGTCCCTGAACTGCTGGGCTCCAGCATGCCACTGTGTCC
CATCGATGAAGCCATCGACAAGAAGATCAAACAAGACTTCAACTCCCTGT
TTCCAAATGCAATAAAGAACATTGGCTTAAATTGCTGGACAGTGTCCTCC
AGAGGGAAGTTGGCCTCCTGCCCAGAAGGCACAGCAGTCTTGAGCTGCTC
CTGTGGCTCTGCCTGTGGCTCGTGGGAC

There is moderate signal that is specific to adipocytes; this signal is present in mesenteric fat and interstitial fat in the neck around the trachea. The expression pattern appears to be specific for adult fat. Further studies are planned to evaluate expression in other anatomical locations of adipose tissue. Brown fat will also be evaluated.

In summary, Murine FIZZ1, FIZZ2 and FIZZ3 have distinct expression patterns. The increased expression of m-FIZZ1 in inflamed pulmonary mucosa and its ability to stimulate the MLR suggest that m-FIZZ1 may function to enhance the mucosal immune response in the lung.

### Example 5

Isolation of cDNAs Encoding m-FIZZ2 and m-FIZZ3

A public expressed sequence tag (EST) DNA databases (Merck/Washington University) was searched with the full-length murine m-FIZZ1 DNA (DNA 53517), and two ESTs, designated AA245405 [FIG. 17, SEQ ID NO: 21) and W42069 (FIG. 18, SEQ ID NO: 22) were identified which showed homology with the m-FIZZ1 DNA.

EST clones AA245405 and W42069 were purchased from Incyte (Palo Alto, Calif.), and sequenced in entirety.

The entire nucleotide sequence of the AA245405 clone is shown in FIG. 9 (SEQ ID NO: 13). This clone, designated DNA 54229, contains a single open reading frame with an apparent translational initiation site at nucleotide positions 106-108 (FIG. 9; SEQ ID NO: 13). The predicted polypeptide precursor (including a signal sequence of 20 amino acids) is 105 amino acids long. Based upon its homology to m-FIZZ1 (51%, using the ALIGN software), the protein was designated m-FIZZ2. Clone DNA54229-1366 has been deposited with ATCC on Apr. 23, 1998 and is assigned ATCC deposit no. 209803.

The entire nucleotide sequence of the W42069 clone is shown in FIG. 11 (SEQ ID NO: 15). This clone, designated DNA 54229, contains a single open reading frame with an apparent translational initiation site at nucleotide positions 75-77 (FIG. 11; SEQ ID NO: 15). The predicted polypeptide precursor (including a signal sequence of 10 amino acids) is 114 amino acids long. Based on its homology to m-FIZZ1 (34%, using the ALIGN software) the protein was designated m-FIZZ3. Clone DNA54231-1366 has been deposited with ATCC on Apr. 23, 1998 and is assigned ATCC deposit no. 209804.

### Example 6

Isolation of DNA Encoding h-FIZZ1

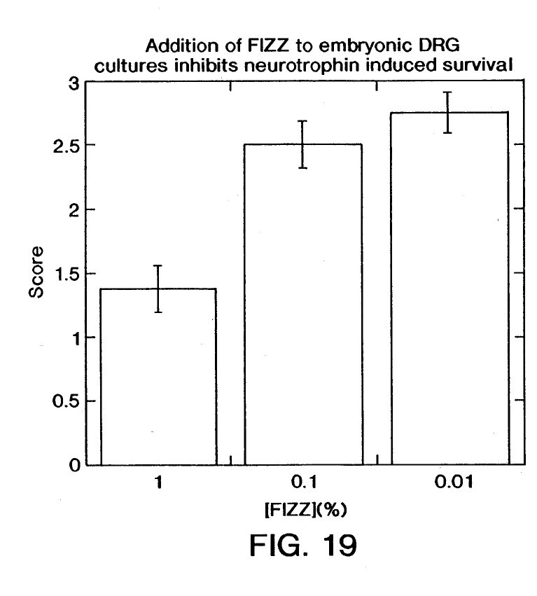

A public expressed sequence tag (EST) DNA database (Merck/Washington University) was searched with the full-length murine m-FIZZ1 DNA (DNA 53517), and an EST, designated AA524300 (FIG. 19, SEQ ID NO: 23), was identified, which showed homology with the m-FIZZ1 DNA.

EST clone AA524300 was purchased from Incyte (Palo Alto, Calif.), and sequenced in entirety.

The entire nucleotide sequence of the AA524300 clone is shown in FIG. 13 (SEQ ID NO: 17). This clone, designated DNA 54228, contains a single open reading frame with an apparent translational initiation site at nucleotide positions 99-101 (FIG. 13; SEQ ID NO: 17). The predicted polypeptide precursor (including a putative signal sequence of 20 amino acids) is 111 amino acids long. Based upon its homology to m-FIZZ1 (50%, using the ALIGN software), the protein is believed to be the human homolog of m-FIZZ1, and is designated h-FIZZ1. Clone DNA54228-1366 has been deposited with ATCC on Apr. 23, 1998 and is assigned ATCC deposit no. 209801.

### Example 7

Isolation of DNA Encoding h-FIZZ3

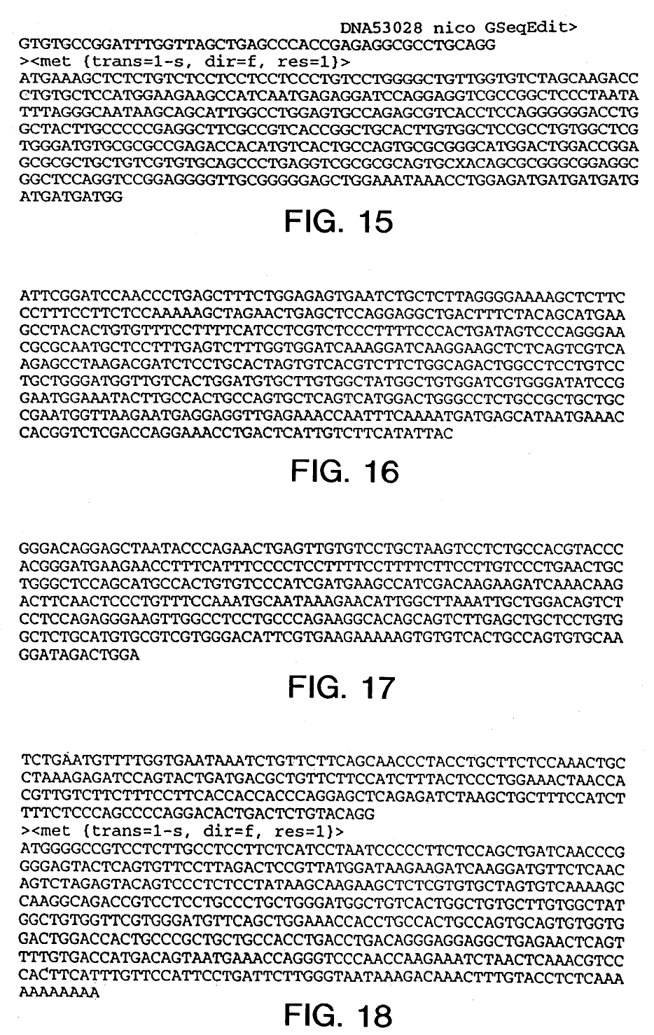

A public expressed sequence tag (EST) DNA database (GenBank) was searched with the full-length murine m-FIZZ1 DNA (DNA 53517), and an EST, designated AA311223 and renamed as DNA53028 (FIG. 15, SEQ ID NO: 19), was identified, which showed homology with the m-FIZZ1 DNA.

Based on the EST sequence, oligonucleotides were synthesized to identify by PCR a cDNA library that contained the sequence of interest and for use as probes to isolate a clone of the full-length coding sequence for h-FIZZ3.

A pair of PCR primers (forward and reverse) and a probe were synthesized:

| | | |
|---|---|---|
| forward primer (h-FIZZ3.f): | GGATTTGGTTAGCTGAGCCCACCGAGA | (SEQ ID NO: 25) |
| reverse primer (h-FIZZ3.r): | GCACTGCGCGCGACCTCAGGGCTGCA | (SEQ ID NO: 26) |
| probe (h-FIZZ3.p): | CTTATTGCCCTAAATATTAGGGAGCCGGCGACCTCCTGGATCCTCTCATT | (SEQ ID NO: 27) |

In order to screen several libraries for a source of a full-length clone, DNA from the libraries was screened by PCR amplification with the PCR primer pair identified above. A positive library was then used to isolate clones encoding the hFIZZ-3 gene using the probe oligonucleotide and one of the PCR primers.

mRNA was isolated from human bone marrow tissue using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to 3-4 kb and the SalI/NotI linkered cDNA was cloned into XhoI/NotI cleaved vector. pRK5D is a cloning vector that has an sp6 transcription initiation site followed by an SfiI restriction enzyme site preceding the XhoI/NotI cDNA cloning sites.

DNA sequencing of the clones isolated as described above gave the full-length DNA sequence for h-FIZZ3 (DNA65351) and the derived protein sequence for PRO1199 (UNQ: 612). A cDNA clone was sequenced in entirety. The entire nucleotide sequence of hFIZZ-3 is shown in FIG. 25 (SEQ ID NO: 23). Clone DNA65351 contains a single open reading frame with an apparent translational initiation site at nucleotide positions 25-27 (FIG. 25; SEQ ID NO: 23). The predicted polypeptide precursor is 108 amino acids long. N-terminal amino acids 1-18 represent a putative signal peptide, and starting at position 57 we have identified a cell attachment sequence motif (RGD). Clone DNA65351 has been deposited with ATCC on May 12, 1998 and is assigned ATCC deposit no. 209856.

### Example 8

Use of FIZZ DNA as a Hybridization Probe

The following method describes use of a nucleotide sequence encoding a FIZZ protein as a hybridization probe.

DNA comprising the coding sequence of a murine or human FIZZ protein is employed as a probe to screen for homologous DNAs (such as those encoding naturally-occurring variants of any of the FIZZ proteins disclosed herein) in murine or human tissue cDNA libraries or murine or human tissue genomic libraries.

Hybridization and washing of filters containing either library DNAs is performed under the following high stringency conditions. Hybridization of radiolabeled FIZZ-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence FIZZ can then be identified using standard techniques known in the art.

### Example 9

Expression of FIZZ Polypeptides in E. coli

This example illustrates preparation of an unglycosylated form of a murine or human FIZZ polypeptide by recombinant expression in E. coli.

The DNA sequence encoding is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the FIZZ coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized FIZZ protein can then be purified using a metal chelating column under conditions that allow tight binding of the protein.

### Example 10

Expression of FIZZ Polypeptides in Mammalian Cells

This example illustrates preparation of a glycosylated form of FIZZ polypeptides by recombinant expression in mammalian cells.

The vector, pRK5 (see EP 307,247, published Mar. 15, 1989), is employed as the expression vector. Optionally, the FIZZ DNA is ligated into pRK5 with selected restriction enzymes to allow insertion of the FIZZ DNA using ligation methods such as described in Sambrook et al., supra. The resulting vector is called pRK5-FIZZ.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-FIZZ DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of FIZZ polypeptide. The cultures containing transfected cells may undergo further incubation (in serum free medium) and the medium is tested in selected bioassays.

In an alternative technique, a FIZZ polypeptide encoding DNA may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-FIZZ DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed FIZZ can then be concentrated and purified by any selected method, such as dialysis and/or column chromatography.

In another embodiment, FIZZ polypeptides can be expressed in CHO cells. The pRK5-FIZZ can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of the FIZZ polypeptide, the culture medium may be replaced with serum free medium. Preferably, the cultures are incubated for about 6 days, and then the conditioned medium is harvested. The medium containing the expressed FIZZ can then be concentrated and purified by any selected method.

Epitope-tagged FIZZ may also be expressed in host CHO cells. The FIZZ polypeptide DNA may be subcloned out of the pRK5 vector. The subclone insert can undergo PCR to fuse in frame with a selected epitope tag such as a poly-his tag into a Baculovirus expression vector. The poly-his tagged FIZZ DNA insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged FIZZ protein can then be concentrated and purified by any selected method, such as by Ni²⁺-chelate affinity chromatography.

### Example 11

Expression of FIZZ Polypeptides in Yeast

The following method describes recombinant expression of FIZZ polypeptides in yeast.

First, yeast expression vectors are constructed for intracellular production or secretion of a desired FIZZ from the ADH2/GAPDH promoter. DNA encoding the FIZZ polypeptide, a selected signal peptide and the promoter is inserted into suitable restriction enzyme sites in the selected plasmid to direct intracellular expression of FIZZ. For secretion, DNA encoding the FIZZ can be cloned into the selected plasmid, together with DNA encoding the ADH2/GAPDH promoter, the yeast alpha-factor secretory signal/leader sequence, and linker sequences (if needed) for expression of FIZZ.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant FIZZ can subsequently be isolated and purified by removing the yeast cells from the fermentation medium by centrifugation and then concentrating the medium using selected cartridge filters. The concentrate containing the desired FIZZ polypeptide may further be purified using selected column chromatography resins.

### Example 12

Expression of FIZZ Polypeptides in Baculovirus Expression System

The following method describes recombinant expression of FIZZ polypeptides in Baculovirus infected insect cells.

The DNA encoding the desired FIZZ polypeptide is fused upstream of an epitope tag contained with a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the FIZZ DNA or the desired portion of the FIZZ DNA (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression is performed as described by O'Reilley et al., Baculovirus expression vectors: A laboratory Manual, Oxford: Oxford University Press (1994).

Expressed poly-his tagged FIZZ can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% Glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% Glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% Glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or western blot with Ni²⁺-NTA-conjugated to alkaline phosphatase (Qiagen). Fractions containing the eluted His₁₀-tagged FIZZ are pooled and dialyzed against loading buffer.

Alternatively, purification of the IgG tagged (or Fc tagged) FIZZ can be performed using known chromatography techniques, including for instance, Protein A or protein G column chromatography.

### Example 13

Rat Dorsal Root Ganglia (RDG) Neuronal Survival Inhibition Assay

Materials ASY Matrix: F12 medium (GIBCO) with the following additives per 100 mls—Sato mix (2.2 ml), PenStrep (1 ml), transferrin (10 μl), insulin (10 μl). Sato mix: 35% BSA (Path-O-Cyte 4, 20 mls), progesterone (0.2 mls, Sigma, P8783, 0.62 mg/ml in EtOH), putrescine (20 ml, Sigma, P7505, 1.61 mg/ml in H₂O), L-Thyroxine (2 mls, Sigma, T0397, 0.4 mg/ml in EtOH), Na Selenite (0.2 mls, Sigma, S9133, 0.387 mg/m; in PBS), Tri-iodo-thyronine (2 mls, Sigma, T6397, 0.337 mg/ml).

Protocol Neural cells (heterogeneous population), freshly isolated from E14 rat embryo dorsal ganglia, were diluted in F12 complete medium, plated at 5,000 cells/well on polyornithine pretreated plates containing 50 μl F12 complete media. Test sample were added in a total of 100 μl of additional medium. After 3 days incubation at 37° C., cell viability was assessed.

Results We have assessed the ability of m-FIZZ proteins to interfere with neurotrophin biological activity using a number of assay systems. The paradigmatic neurotrophin effect is enabling the survival of certain populations of embryonic neurons. Neurotrophins also have effects on adult neurons, although in many cases their present is no longer required to maintain the survival of these cells. As an example, cultures of sensory neurons from embryonic dorsal root ganglia (DRG) are a classical system in which to study neurotrophin action. There are several subpopulations of neurons in these ganglia, and they respond differentially to different members of the neurotrophin family (Snider, Cell 77:627-638 [1994]). Indeed, this assay system is very similar to the one that was used to first purify NGF, the first known member of the neurotrophin family (Cohen, J. Biol. Chem. 234:1129-1137 [1959]; Cohen, Proc. Natl. Acad. Sci. USA 40:1014-1018 [1960]). However, cells from the adult DRG do not require any neurotrophin to survive (Lindsay, J. Neurosci. 8:2394-2405 [1988]), although they clearly still do respond to neurotrophins, both in vivo (Munson et al., J. Neurosci. 17:470-476 [1997]) and in vitro (Lindsay et al., Neurosci. 33:53-65 [1989]).

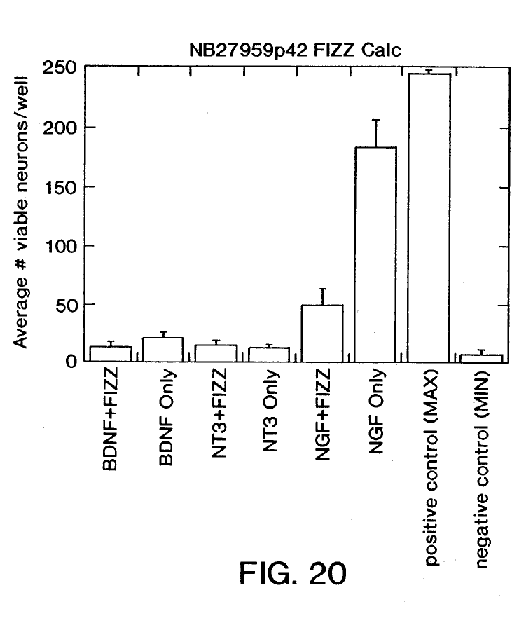

Inclusion of m-FIZZ1 at a concentration of 1 μg/ml in E14 rat embryo dorsal ganglia (RDG) cultures resulted in a significant inhibition of the neuronal survival normally seen in these cultures in the presence of 10 ng/ml each of NGF, BDNF, and NT3. This effect was dose dependent (FIG. 19). m-FIZZ1 inhibited the survival of not only the cells treated with the neutrophin combination, but also the survival induced by NGF alone or BDNF alone (FIG. 20). It was not possible to assess the effect on NT3 induced survival in these experiments.

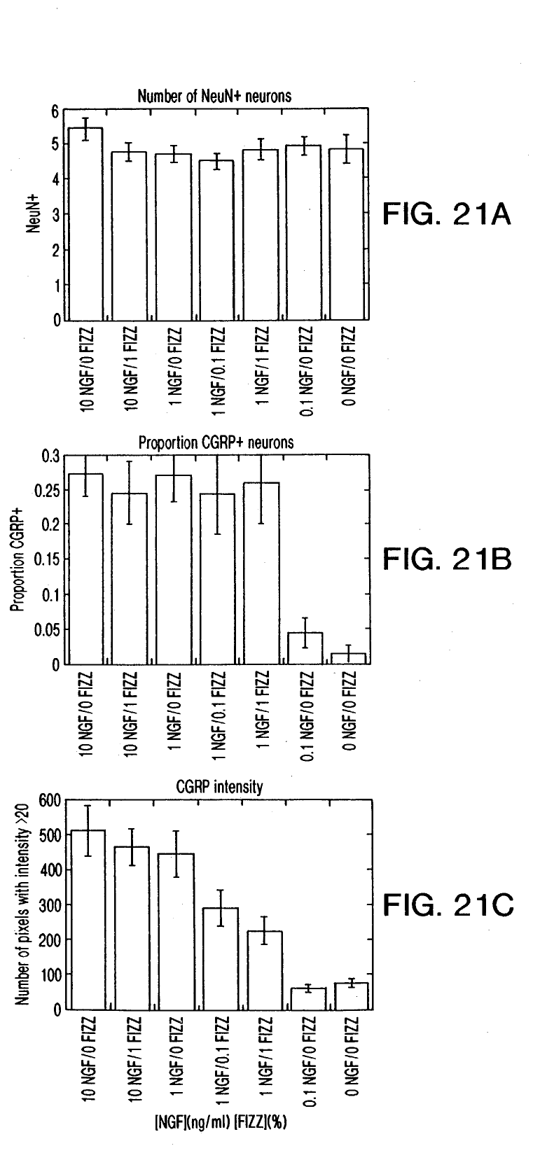

In order to assess whether the neuronal survival inhibitory effect would also be seen in cultures of adult DRG neurons, where neurotrophins are no longer required for survival, we used NGF induced rise in CGRP content as a measure of neurotrophin bioactivity (Lindsay, 1988, supra). Inclusion of NGF in these cultures increases CGRP-like immunoreactivity as previously reported (FIG. 21). m-FIZZ1 is capable of inhibiting this NGF bioactivity in a dose-dependent manner. m-FIZZ1 at a concentration of 1 μg/ml leads to a 50% decrease in CGRP content at 1 ng/ml of NGF concentration. There is no indication of cell death or lack of neurite outgrowth in these cultures, indicating that the observed effect is not due to a general toxic effect of m-FIZZ1. Further, addition of NGF to a concentration of 10 ng/ml is capable of overwhelming the m-FIZZ1 induced inhibition, confirming that this is not merely a toxic effect.

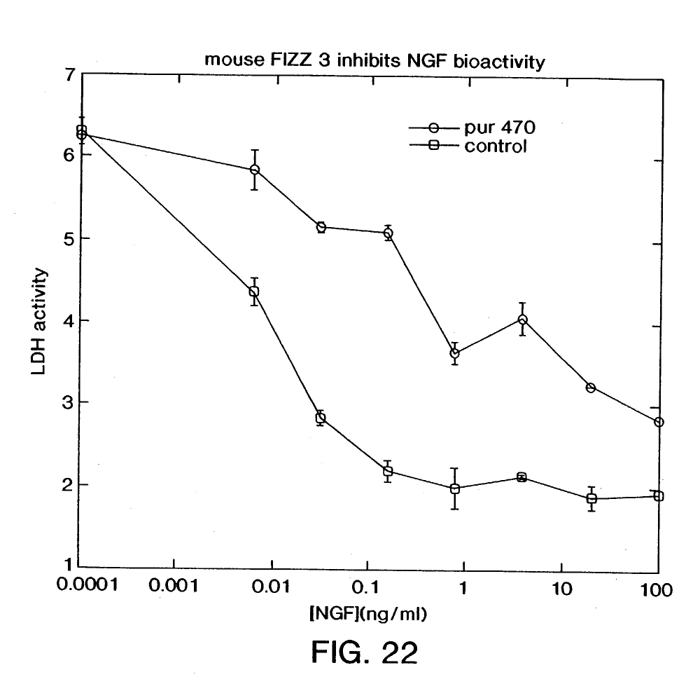

Another biological activity of NGF is the ability to support survival of PC12 cells in serum free media (Rukenstein et al., J. Neurosci. 11:2552-2563 [1991]). The survival can easily be monitored by measurement of LDH release into the medium. As can be seen from FIG. 22, NGF increased cell survival and therefore decreased the LDH release. Inclusion of the mouse 3 form of FIZZ (m-FIZZ3), inhibits this NGF induced survival. This indicates that the homolog of the initially isolated mouse protein shares a similar activity, inhibition of NGF bioactivity.

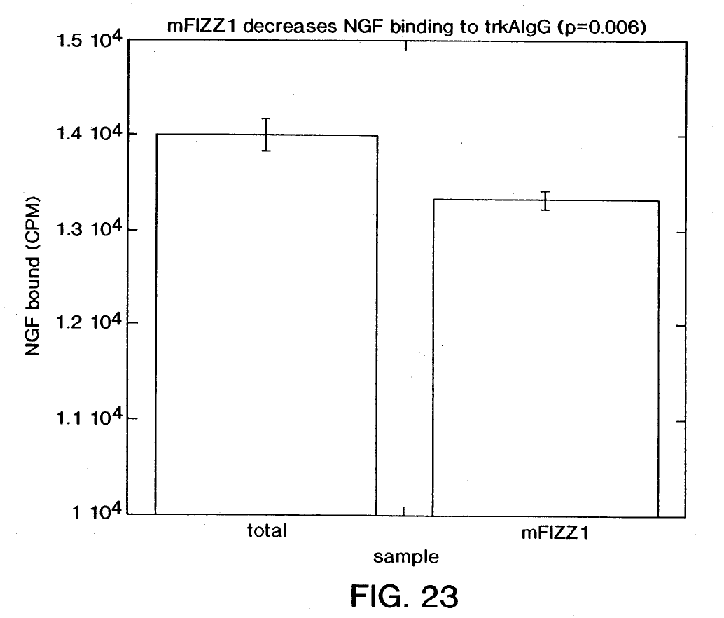

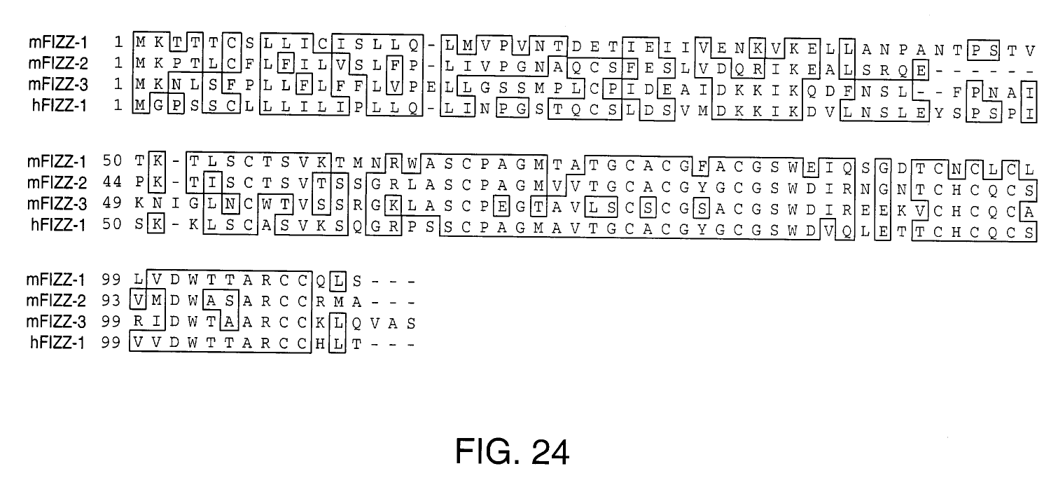

In order to investigate whether this effect might be due to a direct inhibition of NGF binding to its signal transducing receptor trkA, we assessed binding of radiolabeled NGF to trkA-IgG chimeras as previously described (Shelton et al., J. Neurosci. 15:477-491 [1995]). At concentrations of FIZZ of 1 μg/ml, there was a slight but significant decrease of labeled NGF binding observed FIG. 23). An excess of unlabeled NGF and rat or human trkA-IgG did inhibit this binding, indicating that it was specific. This may indicate that m-FIZZ1 might not work only through a direct interaction with trkA/NGF binding but also perhaps with another cell surface receptor present on DRG neurons.

Discussion The primary finding of these experiments is that m-FIZZ1 is capable of inhibiting the actions of neutrophins on responsive neurons. This has been demonstrated for both the survival effect of neurotrophins seen in embryonic DRG neurons and the CGRP upregulation seen with NGF in adult DRG neurons. This effect is not likely to be due to cell toxicity, as it does not cause death of adult neurons, and the inhibition can be overcome with excess NGF. This activity may be due, at least in part, to direct disruption of the trk-neurotrophin interaction, as there was a small, but significant, effect on NGF trk A binding. This activity is likely to be a common action of members of the FIZZ family as at least on other member, mouse FIZZ3 (m-FIZZ3) seems to have similar actions.

The finding that m-FIZZ1 can inhibit various actions of neurotrophins on neurons is important in several respects. First, it is the first description of any endogenous inhibitor of neurotrophin action, and, as such, opens up a new understanding of the possible modulation of neurotrophin activity. In addition to their well known role in development, neurotrophins, or deficiencies of neurotrophins, have been implicated in a number of pathological states. A further understanding of this mode of neurotrophin activity regulation may be crucial to the understanding and treatment of diseases as diverse as asthma, diabetes, inflammation, chronic pain, neuropathy, hypertension, sudden cardiac death, bowel disease, cystitis, and neurodegenerative diseases, such as, Alzheimer's, Parkinson's, Huntington's, Amyotrophic Lateral Sclerosis, and others.

Neurotrophins are now known to control a number of aspects of the function of the peripheral nervous system. The peripheral nervous system, in turn, is capable of modulating the function of essentially all other organ systems. For example, it is now clear that increases in NGF during inflammation increase the sensitivity of primary nociceptors and this is largely responsible for inflammatory pain (McMahon et al., Nature Med. 1:774-780 [1995]; Woolf et al., Neurosci. 62:327-331 [1994]). It is also clear that normal levels of NGF contribute to the maintenance of normal pain sensitivity (McMahon et al., supra). But these sensory nerve fibers contribute to much more than pain sensitivity. They are crucial for normal airway responsiveness, and their removal leads to a lack of normal or pathological modulation of airway constriction. Likewise, upregulation of sensitivity of sensory nerve fibers leads to hyperreflexia in urinary bladder (Dmitrieva et al., Neurosci. 78:449-459 [1997]). Neurotrophins are also known to affect sympathetic neurons, also crucially involved in pain responses (Kinnman and Levine, Neurosci. 64:751-767 [1995]) as well as airway responsiveness, vascular tone, bowel motility, and cardiac rhythm. It has been recently demonstrated that neurotrophins are crucial for the maintenance of normal function in adult motorneurons, as well (Munson et al., 1997, supra), which control all voluntary movement.

In the future, it should be possible to further understand the role of FIZZ in various normal and pathological states by a variety of experiments. First, increased expression of FIZZ will be analyzed in various tissues from patients with conditions indicative of altered nerve function, such as, but not limited to, neuropathy, ALS, impotence, hypertension, chronic pain, asthma, cystitis, bowel disease, cardiac arthythmias, sudden cardiac death, wound healing, stroke, head trauma, vasogenic edema, encephalitis, or CNS degenerative disease. Any decrease or increase in expression in these states may indicate an involvement of FIZZ in the disease process, and so indicate a possible therapeutic role of increasing or decreasing FIZZ activity.

In order to test some specific potential roles of FIZZ, various animal models can be used to explore the consequences of increasing or decreasing FIZZ activity. Animal models of asthma, inflammatory bowel disease, inflammatory pain, cystitis, diabetic neuropathy can all be used in this regard.

### Example 14

Preparation of Antibodies that Bind a FIZZ Polypeptide

This example illustrates preparation of monoclonal antibodies which can specifically bind a FIZZ polypeptide.

Techniques for producing the monoclonal antibodies are known in the art and are described, for instance, in Goding, supra. Immunogens that may be employed include purified FIZZ, fusion proteins containing FIZZ, and cells expressing recombinant FIZZ on the cell surface. Selection of the immunogen can be made by the skilled artisan without undue experimentation.

Mice, such as Balb/c, are immunized with the FIZZ immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-FIZZ antibodies.

After a suitable antibody titer has been detected, the animals “positive” for antibodies can be injected with a final intravenous injection of FIZZ. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against the FIZZ polypeptide. Determination of “positive” hybridoma cells secreting the desired monoclonal antibodies against FIZZ is within the skill in the art.

The positive hybridoma cells can be injected intraperitoneally into syngeneic Balb/c mice to produce ascites containing the anti-FIZZ monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 15

Mixed Lymphocyte Reaction (MLR) Stimulation Assay

The Mixed Lymphocyte Reaction (MLR) assay evaluates CD4+ T lymphocyte function, more specifically, the ability of T lymphocytes to proliferate in response to the presentation of allo-antigen. In the one-way MLR assay, a donor population of peripheral blood mononuclear cells (PBMCs) is challenged with an irradiated stimulator population of PBMCs. The antigen to which the T lymphocytes respond is a mismatched MHC molecule that is expressed and presented by antigen presenting cells in the stimulator population. The assay identifies molecules that either enhance or inhibit the proliferation of the responder T lymphocyte in response to stimulation with presented allo-antigen.

Molecules that enhance (stimulate) MLR response enhance or potentiate the immune response to antigen. Accordingly, such molecules (or small molecule or antibody agonists of such molecules) are candidates for the treatment of conditions where the enhancement of the immune response would be beneficial. In addition, inhibitors of such stimulatory molecules may be useful where suppression of the immune response would be of value. For example, using neutralizing antibodies or small molecule antagonists that inhibit the molecules with stimulatory activity in the MLR could be beneficial in the treatment of immune-mediated inflammatory diseases. Molecules that inhibit the MLR (or their small molecule or antibody agonists) could be useful in inhibiting the immune response and that ameliorating immune-mediated diseases.

Frozen PBMCs were thawed and cultured in RPMI+10% FBS the night before wash. The cells were resuspended in RPMI+10% FBS at a concentration of 3×10⁶ cells/ml. 100 μl of the cell suspension were incubated at 37° C., 5% CO, with 100 μl of test samples of murine and human FIZZ1, respectively, at concentrations shown in the following Tables 1 and 2. On the fifth day, the cells were pulsed for six hours then harvested.

In a first set of experiments, the effect of murine and human FIZZ1-IgG fusions on human T lymphocyte (PBMC) proliferation was tested at various concentrations. The results are shown in Table 1. In a second experiment, the effect of various concentrations of a murine FIZZ1-IgG fusion on the proliferation of murine PBMC's was tested. The results are shown in Table 2.

The results are expressed as the percent increase (SI) over the control (the responder and stimulator cells together, that represents the maximum of the assay with no additional stimulation), that is set at 100%.

TABLE 1

| Test compound | Concentration (%) | SI (%) |
|---|---|---|
| mouse-FIZZ1-IqG | 5 | 228* |
| | 1 | 253* |
| | 0.10 | 228* |
| | 0.01 | 118 |
| human FIZZ1-IgG | 5 | 152* |
| | 1 | 81 |
| | 0.10 | 113 |
| | 0.01 | 95 |

TABLE 2

| Test compound | Concentration (%) | SI (%) |
|---|---|---|
| mouse-FIZZ1-IgG | 5.00 | 215* |
| | 2.50 | 102 |
| | 1.25 | 138 |
| | 0.63 | 120 |
| | 0.31 | 81 |
| | 0.16 | 52 |
| | 0.08 | 86 |

The results marked by asterisk (*) demonstrate that murine and human FIZZ1 stimulate the MLR as measured by the increased T cell proliferation. (In similarly conducted MLR assays, FIZZ2 and FIZZ3 stimulated the MLR as measured by increased T cell proliferation (data not shown)). The ability of murine FIZZ1 (m-FIZZ1) to stimulate the MLR was further verified by using an anti-m-FIZZ1 polyclonal antibody. While the addition of the antibody alone to the MLR did not significantly affect proliferation, the antibody blocked the ability of m-FIZZ1 to stimulate MLR. This result further verifies that m-FIZZ1 indeed stimulates the MLR.

Deposit of Material

The following materials have been deposited with the American Type Culture Collection, 10801 University Blvd., Manassas, Va. 20110-2209 (ATCC):

| Material | ATCC Dep. No. | Deposit Date |
|---|---|---|
| DNA 53517-1366 | 209802 | Apr. 23, 1998 |
| DNA 54229-1366 | 209803 | Apr. 23, 1998 |
| DNA 54231-1366 | 209804 | Apr. 23, 1998 |
| DNA 54228-1366 | 209801 | Apr. 23, 1998 |
| DNA 65351-1366-1 | 209856 | May 12, 1998 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. A method of treating a pathologic condition associated with neurotrophin action on responsive neurons, comprising
administering to a mammal an effective amount of a FIZZ protein or an agonist of a FIZZ protein.

2. The method of claim 1 wherein said FIZZ protein comprises amino acid residues 24-117 of FIG. 5 (SEQ ID NO: 10).

3. The method of claim 1 wherein said FIZZ protein comprises amino acid residues 21-105 of FIG. 10 (SEQ ID NO: 14).

4. The method of claim 1 wherein said FIZZ protein comprises amino acid residues 21-114 of FIG. 12 (SEQ ID NO: 16).

5. The method of claim 1 wherein said FIZZ protein comprises amino acid residues 21-111 of FIG. 14 (SEQ ID NO: 18).

6. The method of claim 1 wherein said FIZZ protein comprises amino acid residues 19-108 of FIG. 26 (SEQ ID NO: 24).

7. A method of treating a pathologic condition associated with the neurotrophin-inhibitory activity of a FIZZ polypeptide, comprising administering to a mammal an antagonist of a FIZZ protein.

8. The method of claim 7 wherein said antagonist is an anti-FIZZ antibody.

9. A method of screening for an antagonist of a FIZZ polypeptide, comprising contacting neurotrophin-responsive neurons, in the presence of a neurotrophin and a FIZZ polypeptide, with a candidate molecule, and monitoring neurotrophin action on the neurons.

10. A method of screening for an agonist of a FIZZ polypeptide, comprising contacting neurotrophin-responsive neurons, in the presence of a neurotrophin, with a candidate molecule, and monitoring neurotrophin action on the neurons.

11. A method of enhancing the immune response in a patient comprising administering to said patient an effective amount of a FIZZ protein or an agonist of a FIZZ protein.

12. The method of claim 11 wherein said FIZZ protein comprises
amino acid residues 24-117 of FIG. 5 (SEQ ID NO: 10).

13. The method of claim 11 wherein said FIZZ protein comprises
amino acid residues 21-105 of FIG. 10 (SEQ ID NO: 14).

14. The method of claim 11 wherein said FIZZ protein comprises
amino acid residues 21-114 of FIG. 12 (SEQ ID NO: 16).

15. The method of claim 11 wherein said FIZZ protein comprises
amino acid residues 21-111 of FIG. 14 (SEQ ID NO: 18).

16. The method of claim 11 wherein said FIZZ protein comprises
amino acid residues 19-108 of FIG. 26 (SEQ ID NO: 24).

17. A method of suppressing the immune response in a mammal comprising administering to said mammal an effective amount of an antagonist of a FIZZ protein.

18. The method of claim 17 wherein said antagonist is an anti-FIZZ antibody.

19. A composition comprising a FIZZ polypeptide, or an agonist or antagonist of a FIZZ polypeptide, in combination with a pharmaceutically acceptable carrier.
